(19) United States
(12) Patent Application Publication
(10) Pub. No.: US 2021/0363522 A1
(43) Pub. Date: Nov. 25, 2021

(54) CRISPR/CAS-RELATED METHODS AND COMPOSITIONS TARGETING VIRUS GENOMES

(71) Applicant: EDITAS MEDICINE, INC., Cambridge, MA (US)

(72) Inventors: Benjamin Aryeh DINER, Somerville, MA (US); Deepak REYON, Somerville, MA (US)

(73) Assignee: EDITAS MEDICINE, INC., Cambridge, MA (US)

(21) Appl. No.: 17/388,707

(22) Filed: Jul. 29, 2021

Related U.S. Application Data
(63) Continuation of application No. PCT/US2020/016112, filed on Jan. 31, 2020.
(60) Provisional application No. 62/800,231, filed on Feb. 1, 2019.

(51) Int. Cl. C12N 15/11; C12N 9/22

(52) U.S. Cl. CPC C12N 15/111; C12N 2320/30; C12N 2310/20; C12N 9/22

(57) ABSTRACT

The present disclosure relates to compositions, systems, vectors, and methods for the treatment, prevention, and/or reduction of viral infection and viral infection-related diseases. In particular, the methods disclosed herein involve gene editing approaches using a genome editing system targeting a viral genome, where the expression of at least one component of the gene editing system is regulated by a promoter derived from the targeted viral family, genus, and/or species.

### CROSS-REFERENCE TO RELATED APPLICATIONS

This application is a continuation of International Patent Application No. PCT/US2020/016112, filed on Jan. 31, 2020, which claims priority to U.S. Provisional Application No. 62/800,231 filed on Feb. 1, 2019, the contents of which are incorporated by reference in their entireties, and to which priority is claimed.

### SEQUENCE LISTING

This application contains a Sequence Listing, which was submitted in ASCII format via EFS-Web, and is hereby incorporated by reference in its entirety. The ASCII copy, created on Jul. 29, 2021, is named 084177 0252 SL.txt and is 11,263 bytes in size.

### FIELD

The present disclosure relates to CRISPR/Cas-related methods and components for editing a virus genome, or modulating expression of a virus genome, and applications thereof in connection with treating, preventing, and/or reducing viral infections and viral infection-related diseases.

### BACKGROUND

CRISPRs (Clustered Regularly Interspaced Short Palindromic Repeats) evolved in bacteria and archaea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a Cas9 protein to a target sequence in the viral genome. The Cas9 protein, in turn, cleaves and thereby silences the viral target.

Recently, the CRISPR/Cas system has been adapted for genome editing in eukaryotic cells. The introduction of site-specific double strand breaks (DSBs) allows for target sequence alteration through endogenous DNA repair mechanisms, for example non-homologous end-joining (NHEJ) or homology-directed repair (HDR).

Gene editing can be used to disrupt viral gene function and limit viral replication and spread. However, weak or inappropriate expression of gene editing components in cells compromises therapeutic efficacy or safety. The existing promoters that have been used with CRISPR/Cas9 have many issues. For example, some promote are not resistant to viral-dependent cellular gene silencing; some promoters have strong expression throughout the temporal cascade of the viral gene expression, and thus raise off-target concerns; and some promoters are tissue-specific and do not have any activity in certain tissues where gene editing is desired.

### SUMMARY

Provided herein are compositions, systems, vectors, and methods for the treatment, prevention, and/or reduction of viral infections and viral infection-related diseases. In certain embodiments, the methods disclosed herein involve gene editing approaches using a genome editing system targeting the viral genome, and a promoter derived from a genome of the virus, where the expression of at least one component of the gene editing system is regulated by the promoter.

In one aspect, the present disclosure relates to a genome editing system including: (a) an RNA-guided nuclease, and (b) a gRNA molecule including a targeting domain that is complementary with a target sequence of a target gene of a targeted virus; wherein the expression of (a) and/or (b) is regulated by a promoter that is derived from a genome of the family, genus, or species of the targeted virus. In another aspect, the presently disclosed subject matter relates to a composition including (a) an RNA-guided nuclease, and (b) a gRNA molecule including a targeting domain that is complementary with a target sequence of a target gene of a targeted virus; wherein the expression of (a) and/or (b) is regulated by a promoter that is derived from a genome of the family, genus, or species of the targeted virus. In another aspect, the presently disclosed subject matter relates to a vector including a polynucleotide encoding (a) an RNA-guided nuclease, and (b) a gRNA molecule including a targeting domain that is complementary with a target sequence of a target gene of a virus; wherein the expression of (a) and/or (b) is regulated by a promoter that is derived from a genome of the virus. In another aspect, the present disclosure relates to a genome editing system, including (a) an RNA-guided nuclease; and (b) a gRNA molecule comprising a targeting domain that is complementary with a target sequence of a target gene of a targeted virus; wherein when the genome editing system is introduced in a cell infected by the targeted virus, the expression of the gene editing system correlates with transcriptional activity of the targeted virus, and/or genome abundance of the targeted virus.

In various non-limiting embodiments, the promoter is derived from a gene of the family, genus, or species of the targeted virus.

In various non-limiting embodiments, the promoter is operably linked to a polynucleotide encoding (a), and/or a polynucleotide encoding (b).

In various non-limiting embodiments, the promoter is derived from an immediate early gene, an early gene, or a late gene of the family, genus, or species of the targeted virus.

In various non-limiting embodiments, the expression of (a) and/or (b) is weak during a viral latency. In various non-limiting embodiments, the expression of (a) and/or (b) is strong during a viral reactivation. In various non-limiting embodiments, the expression of (a) and/or (b) during the viral latency is at a level at least about 10%, at least about 20%, at least about 30%, at least about 40% or at least about 50% lower than the expression of (a) and/or (b) during the viral reactivation.

In various non-limiting embodiments, the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Epstein-Barr virus, and a Kaposi's sarcoma-associated herpesvirus. In various non-limiting embodiments, the targeted virus is selected from the group consisting of a Simplexvirus, a Varicellovirus, a Cytomegalovirus, a Roseolovirus, a Lymphocryptovirus, and a Rhadinovirus.

In various non-limiting embodiments, the targeted virus is a Herpes Simplex Virus (HSV).

In various non-limiting embodiments, the targeted virus is a Herpes Simplex Virus-1 (HSV-1).

In various non-limiting embodiments, the RNA-guided nuclease is a Cas9 molecule. In various non-limiting embodiments, the Cas9 molecule comprises an S. pyogenes Cas9 molecule or an S. aureus Cas9 molecule. In various non-limiting embodiments, the Cas9 molecule comprises a wild-type Cas9 molecule, a mutant Cas9 molecule, or a combination thereof. In various non-limiting embodiments, the mutant Cas9 molecule comprises a D10A mutation.

In various non-limiting embodiments, the RNA-guided nuclease is a Cpf1 molecule.

In various non-limiting embodiments, the gRNA molecule is modified at its 5′ end. In various non-limiting embodiments, the modification comprises an inclusion of a 5′ cap. In various non-limiting embodiments, the 5′ cap comprises a 3′-O-Me-m7G(5′)ppp(5′)G anti reverse cap analog (ARCA). In various non-limiting embodiments, the gRNA molecule comprises a 3′ polyA tail that is comprised of about 10 to about 30 adenine nucleotides. In various non-limiting embodiments, the 3′ polyA tail is comprised of 20 adenine nucleotides

In various non-limiting embodiments, the promoter is activated by a transactivator of a genome of the family, genus, or species of the targeted virus. In various non-limiting embodiments, transactivator is selected from a group consisting of a HSV-1 ICP0 protein, a HSV-1 ICP4 protein, and a HSV-1 ICP27 protein.

In another aspect, the presently disclosed subject matter relates to a method of altering a target gene of a targeted virus in a cell, including administrating to the cell one of: (i) a genome editing system including a gRNA molecule including a targeting domain that is complementary with a target sequence of a target gene of the targeted virus, and an RNA-guided nuclease; (ii) a genome editing system including a polynucleotide encoding the gRNA molecule including the targeting domain that is complementary with the target sequence of the target gene of the targeted virus, and a polynucleotide encoding the RNA-guided nuclease; (iii) a composition including the gRNA molecule including the targeting domain that is complementary with the target sequence of the target gene of the targeted virus, and the RNA-guided nuclease; and (iv) a vector including a polynucleotide encoding the gRNA molecule including the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

In another aspect, the presently disclosed subject matter relates to a genome editing system for use in altering a target gene of a targeted virus in a cell, wherein the genome editing system comprising: (i) a gRNA molecule comprising a targeting domain that is complementary with a target sequence of a target gene of the targeted virus, and an RNA-guided nuclease; or (ii) a polynucleotide encoding the gRNA molecule comprising the targeting domain that is complementary with the target sequence of the target gene of the targeted virus, and a polynucleotide encoding the RNA-guided nuclease, wherein the expression of the gRNA molecule and/or the RNA-guided nuclease is regulated by a promoter that is derived from a genome of the family, genus, or species of the targeted virus. In another aspect, the presently disclosed subject matter relates to a composition for use in altering a target gene of a targeted virus in a cell, wherein the composition comprising the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus. In another aspect, the presently disclosed subject matter relates to a vector for use in altering a target gene of a targeted virus in a cell, wherein the vector comprising a polynucleotide encoding the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

In various non-limiting embodiments, the cell is an erythroid cell, or a trigeminal cell.

In various non-limiting embodiments, the one of (i)-(iv) is administered in vivo.

In another aspect, the presently disclosed subject matter relates to a method for treating and/or preventing a virus-related disease in a subject, including administrating to the subject one of: (i) a genome editing system including a gRNA molecule including a targeting domain that is complementary with a target sequence of a target gene of the targeted virus, and an RNA-guided nuclease; (ii) a genome editing system including a polynucleotide encoding the gRNA molecule including the targeting domain that is complementary with the target sequence of the target gene of the targeted virus, and a polynucleotide encoding the RNA-guided nuclease; (iii) a composition including the gRNA molecule including the targeting domain that is complementary with the target sequence of the target gene of the targeted virus, and the RNA-guided nuclease; and (iv) a vector including a polynucleotide encoding the gRNA molecule including the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus. In another aspect, the presently disclosed subject matter relates to a genome editing system for use in treating and/or preventing a virus-related disease in a subject, wherein the genome editing system comprising: (i) a gRNA molecule comprising a targeting domain that is complementary with a target sequence of a target gene of the targeted virus, and an RNA-guided nuclease; or (ii) a polynucleotide encoding the gRNA molecule comprising the targeting domain that is complementary with the target sequence of the target gene of the targeted virus, and a polynucleotide encoding the RNA-guided nuclease, wherein the expression of the gRNA molecule and/or the RNA-guided nuclease is regulated by a promoter that is derived from a genome of the family, genus, or species of the targeted virus. In another aspect, the presently disclosed subject matter relates to a composition for use in treating and/or preventing a virus-related disease in a subject, wherein the composition comprising the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus. In another aspect, the presently disclosed subject matter relates to a vector for use in treating and/or preventing a virus-related disease in a subject, wherein the vector comprising a polynucleotide encoding the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

In various non-limiting embodiments, the administration is initiated at an early stage, a late stage, an advanced stage, a severe stage, or an acute stage of the viral-related disease. In various non-limiting embodiments, the administration is initiated prior to the subject is exposed to the targeted virus. In various non-limiting embodiments, the administration is initiated prior to the virus-related disease onset.

In various non-limiting embodiments, the viral-related disease is a HSV-1 infection.

In various non-limiting embodiments, the subject is a human subject.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure.

FIG. 1 is a graphic representation of a non-limiting exemplary design of an AAV vector that encodes CRISPR/Cas system for targeting HSV.

FIG. 2 shows literature search identifying exemplary HSV-1 promoters. Literature-defined promoter boundary coordinates were identified within HSV-1 strain 17+ (NCBI accession JN555585.1) and extended to include 5′ UTR elements up to the translational start codon.

FIGS. 3A-3B show PCR amplification of HSV-1 promoters and fusion to mCherry reporter gene. (A) Schematic of target HSV-1 promoter fused to mCherry reporter gene followed by a mini poly(A) transcriptional terminator. (B) PCR amplification of HSV-1 promoters from HSV-1 17+ genomic DNA and annealed to mCherry cDNA/poly(A) amplicon using overlap extension PCR.

FIG. 4 shows HSV promoter inducibility by HSV-1 infection. Cells nucleofected with plasmid encoding the indicated HSV promoter fused to mCherry reporter gene were infected with HSV-1-GFP and imaged over 24 hours. Example fluorescent microscopy images of infected cell populations at different hours post-infection (hpi) are shown.

FIGS. 5A-5C show flow cytometry analysis to quantify HSV-dependent HSV promoter inducibility. Indicated promoter-mCherry fusions were nucleofected into cells and then challenged with HSV-1-GFP for 8 hours. PFA-fixed cells were analyzed by flow cytometry based on mCherry- and GFP-positivity. Light gray bar (−HSV; −GFP) represents GFP-negative cells in uninfected condition. Dark gray bar (+HSV; +GFP) represents GFP-positive cells in HSV-infected condition. Averages+/−SD are shown (n=5). IE, immediate early; E, early; L, late promoters. (A) Percent mCherry-positivity. (B) Mean fluorescence intensity (MFI) for mCherry-positive cells. (C) Results of (A) and (B).

FIG. 6 shows HSV-dependent viral promoters mediating CRISPR-based knockdown of HSV replication in culture. Cells nucleofected with plasmid encoding a U6/UL48-directed gRNA cassette and a SaCas9 cassette driven by the indicated HSV promoter were challenged with HSV at an MOI of 0.1. qPCR was used to quantify HSV genomes in the cell culture supernatant after 24 hours post-infection. Averages+/−SD are shown (n=3 biological rep; n=2 technical rep). IE, immediate early; E, early; L, late promoters.

FIG. 7 shows flow cytometry analysis to assess HSV promoter inducibility by HSV-1 infection. Cells nucleofected with plasmid encoding the indicated HSV promoter fused to mCherry reporter gene were infected with HSV-1-GFP for 8 hours. Cells were then fixed with PFA and sorted for mCherry and GFP positivity. Exemplary cell distribution contour plots are shown.

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The phrase “consisting essentially of” means that the species recited are the predominant species, but that other species may be present in trace amounts or amounts that do not affect structure, function or behavior of the subject composition. For instance, a composition that consists essentially of a particular species will generally comprise about 90%, about 95%, about 96%, or more of that species.

“Domain” is used to describe a segment of a protein or nucleic acid. Unless otherwise indicated, a domain is not required to have any specific functional property.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g., a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai et al. (Nat. Biotechnol. 34(5): 483 (2016), incorporated by reference herein) or by other means well known in the art. Genome editing outcomes may also be assessed by in situ hybridization methods such as the FiberComb™ system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g., a nucleic acid or protein), does not require a process limitation but merely indicates that the replacement entity is present.

“Subject” means a human or non-human animal. A human subject can be any age (e.g., an infant, child, young adult, or adult), and may suffer from a disease, or may be in need of alteration of a gene. Alternatively, the subject may be an animal, which term includes, but is not limited to, mammals, birds, fish, reptiles, amphibians, and more particularly non-human primates, rodents (such as mice, rats, hamsters, etc.), rabbits, guinea pigs, dogs, cats, and so on. In certain embodiments of this disclosure, the subject is livestock, e.g., a cow, a horse, a sheep, or a goat. In certain embodiments, the subject is poultry.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

The term “keratitis” or “ocular keratitis” refers to a condition in which the eye's cornea, the clear dome on the front surface of the eye, becomes inflamed. In certain embodiments, the ocular keratitis is HSV-1 ocular keratitis. In certain embodiments, the ocular keratitis is HSV-2 ocular keratitis.

A “Kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g., suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

As used herein, the term “promoter” refers to a region (i.e., a DNA sequence) of a genome that initiates the transcription of a gene.

As used herein, the term “transactivator” refers to a protein that can bind to a promoter and activate the promoter, initiating the transcription of a gene.

### Overview

Disclosed herein are compositions, systems, vectors, and methods for the treatment, prevention, and/or reduction of viral infections and viral infection-related diseases. In certain embodiments, the methods disclosed herein involve gene editing approaches using a genome editing system targeting a viral genome, where the expression of at least one component of the gene editing system is regulated by a promoter derived from the targeted viral family, genus, and/or species.

In one aspect, the subject matter disclosed herein leverages the conditional activation of viral promoters during infection of a host cell to deliver CRISPR/Cas components in a targeted way. Additionally, the subject matter disclosed herein, can, in certain embodiments, limit the expression of CRISPR/Cas components designed to disrupt the genome of a particular family, genus, and/or species of virus to only infected cells which are undergoing productive viral infection. Furthermore, the subject matter disclosed herein can, in certain embodiments, reduce off-target editing of the cellular genome and other unintended detrimental effects to the cell. For example, the subject matter disclosed herein can, in certain embodiments, advantageously mitigate the risk of off-target editing of the host cell genome while maintaining on-target editing of extrachromasomal viral DNA by making gene editing dependent on viral genome expression of viral transactivators.

### Viral Genome Expression Dependent Gene Editing Approaches

Like those encoded within the genomes of their host cell, promoters within the genome of a virus also contain cis-acting elements at the nucleotide level that function to tightly regulate viral gene expression in a conditional or temporal manner. During infection, the viral genome is released into the cellular environment, where cellular or viral transcription factors bind these cis-acting elements and drive the virus through it's replication cycle in order to propagate and spread within it's host. Oftentimes, transcription factors required for viral gene expression (either cellular or viral origin) are only expressed during productive viral replication as a means for controlling the fate of the host cell and providing greater fitness advantage to the virus itself.

For instance, herpes simplex virus-1 (HSV-1), like all other members of the herpesviridae family, has the ability to establish a latent infection in neurons within the head and neck ganglia of humans. During latency, the viral genome is mostly transcriptionally inactive due to the presence of heterochromatin on the genome and absence of HSV-1 transcription factors ICP0, ICP4, and ICP27. However, when the host neuron is stressed by certain external stimuli, induced cellular factors remove inhibitory heterochromatin, expression of ICP0, ICP4, ICP27 and other proteins belonging to the “immediate-early” kinetic class are induced. These “immediate-early” genes function to induce expression of “early” genes, which, in turn, induce expression of “late” genes, allowing replication and spread of the viral genome to neighboring cells.

Recurrent viral infection (e.g., recurrent HSV infect and recurrent HSV ocular keratitis) is a result of latent virus reactivation within the latency tissue (e.g., the trigeminal ganglia (TG)). For example, in both nonneuronal and neuronal cells, virus (e.g., HSV-1, HSV-2, and CMV) can undergo a productive infection. The genes of the virus are transcribed in an ordered cascade. In peripheral neurons, the virus can also establish a more quiescent or latent infection. During latency, the viral gene expression is substantially reduced comparing to the productive infection stage. During the cascade of the productive cycle, the viral gene expression can be divided into three general stages: 1) immediate-early (IE), 2) early (E), and 3) late (L). This cascade results from the interplay between viral and cellular factors (transcriptional and post-transcriptional) and the promoter architectural and structural differences within each of the three gene classes. Virus relies on the essential viral genes (e.g., immediate-early, early and late genes) for infection, proliferation and assembly.

A gene editing approach using a genome editing system (e.g., CRISPR/Cas9) to target viral genomes, e.g., knocking out viral genes (e.g., essential viral genes or non-essential viral genes), individually or in combination can limit viral resistance and treat primary and recurrent viral infections. In certain embodiments, methods described herein include knocking out at least one viral gene. In certain embodiments, the viral gene is an essential viral gene. In certain embodiments, the method comprises knocking out two or more viral genes. In certain embodiments, the method comprises knocking out two viral genes, e.g., two essential viral genes, two non-essential viral genes, or one essential viral gene and one non-essential viral gene. In certain embodiments, the viral gene is an HSV gene, an HSV-1 gene, an HSV-2 gene, or a CMV gene.

In certain embodiments, the expression of at least one component of the genome editing system is regulated by a promoter. The existing promoters that have been used to regulate the expression of CRISPR/Cas9 have many issues. For example, some promoters are not resistant to viral-dependent cellular gene silencing; some promoters have strong expression throughout the temporal cascade of the viral gene expression, and thus raise off-target concerns; and some promoters are tissue-specific and do not have any activity in certain tissues where gene editing is desired. The presently disclosed promoters have, in certain embodiments, the advantages of: 1) being resistant to viral-dependent cellular gene silencing during reactivation, 2) having timed differential expression at latency and reactivation (e.g., weak expression at latency and strong expression at reactivation, and/or 3) only having activity in target tissues (e.g., latency tissues and cells, e.g., trigeminal dorsal root ganglion, the cervical dorsal root ganglia, and the sacral dorsal root ganglia).

Disclosed herein are gene editing systems targeting viral genomes, where, in certain embodiments, the expression of at least one component of the gene editing system is regulated by a promoter derived from the genome of the family, genus, and/or species of virus being targeted. Such a promoter is activated by viral gene expression, and the activated promoter in turn induces expression of the component of the gene editing system under the control of the activated promoter. As a result, at least one component of the gene editing system is only expressed when the genes of the targeted family, genus, and/or species of virus are expressed. For example, after introducing the gene editing system into a cell infected by a virus, the expression of the gene editing system can be modulated by the transcriptional activity of the virus through the promoter, which includes the temporal cascade of viral gene expressions, which itself can be modulated by the interplay between viral and cellular factors (transcriptional and post-transcriptional).

In one aspect, the present disclosure provides gene editing systems comprising (a) an RNA-guided nuclease, and (b) a RNA molecule comprising a targeting domain that is complementary with a target sequence of a target gene of a virus, wherein the expression of (a) and/or (b) is regulated by a promoter derived from a genome of the family, genus, and/or species of the targeted virus.

The present disclosure further provides compositions comprising such gene editing systems, vectors encoding such gene editing systems, and methods for using such gene editing systems.

In certain embodiments, the presently disclosed promoters can be regulated by the transcriptional activity of the targeted virus, and such transcription activity can be further modulated by a number of viral and cellular factors. For example, when the cis-transcriptional activity is high (e.g., during reactivation stage), the viral productive cascade will be triggered. At the same time, the cis-transcriptional activity activates the promoter, and thus induces the expression of at least one component of the genome editing system. As a result, the expression of at least one component of the gene editing system corresponds to the viral transcription activity.

In certain embodiments, the virus is a virus of Herpesviridae family. In certain embodiments, the virus is a virus of Alphaherpesvirinae subfamily, Betaherpesvirinae subfamily, or Gammaherpesvirinae subfamily. In certain embodiments, the virus is an Iltovirus, a Mardivirus, a Simplexvirus, a Scutavirus, or a Varicellovirus. In certain embodiments, the virus is a HSV, HSV-1, or HSV-2. In certain embodiments, the virus is a Cytomegalovirus (CMV), a Morumegalovirus, a Proboscivirus, or a Roseolovirus. In certain embodiments, the virus is a Lymphocryptovirus, a Macavirus, a Percavirus, or a Rhadinovirus. In certain embodiments, the virus is a human herpesvirus, such as a Human Cytomegalovirus (HCMV), a Kaposi Sarcoma-Associated Herpesvirus (KSHV), an Epstein-Barr virus (EBV), and a Varicella-Zoster virus (VZV). In certain embodiments, the virus is a Human Immunodeficiency Virus (HIV) or a Human Papillomavirus (HPV).

In certain embodiments, the promoters are derived from genes (i.e., a gene DNA sequence) of the same family, genus, and/or species of the targeted virus. In certain embodiments, the gene is an immediate-early gene, a late gene, or an early gene. In certain embodiments, the gene is a HSV-1 gene. In certain embodiments, the gene is selected from the group consisting of LAT, RL2, US12, SI, UL54, UL23, UL29, UL39, US6, UL19, UL37, UL27, UL44, and UL38. Non-limiting exemplary promoters that can be used with the present disclosure include SEQ ID NOs: 1-14 as follows:

LAT promoter
[SEQ ID NO.: 1]
CTGCAGACAGGGGCACCGCGCCCGGAAATCCATTAGGCCGCAGACGAGGAAAATAAAATTACATCACC
TACCCACGTGGTGCTGTGGCCTGTTTTTGCTGCGTCATCTCAGCCTTTATAAAAGCGGGGGCGCGGCC
GTGCCGATCGCGGGTGGTGCGAAAGACTTTCCGGGCGCGTCCGGGTGCCGCGGCTCTCCGGGCCCCCC
TGCAG
RL2 promoter
[SEQ ID NO.: 2]
CCCGGGAGCTCCGCACCAAGCCGCTCTCCGGAGAGACGATGGCAGGAGCCGCGCATATATACGCTTGG
AGCCAGCCCGCCCTCACAGGGCGGGCCCGCCTCGGGGGCGGGACTGGCCAATCGGCGGCCGCCAGCGC
GGCGGGGCCCGGCCAACCAGCGTCCGCCGAGTCTTCGGGGCCCGGCCCATTGGGCGGGAGTTACCGCC
CAATGGGCCGGGCCGCCCACTTCCCGGTATGGTAATTAAAAACTTGCAAGAGGCCTTGTTCCGCTTCC
CGGTATGGTAATTAGAAACTCATTAATGGGCGGCCCCGGCCGCCCTTCCCGCTTCCGGCAATTCCCGC
GGCCCTTAATGGGCAACCCCGGTATTCCCCGCCTCCCGCGCCGCGCGTAACCACTCCCCTGGGGTTCC
GGGTTATGCTAATTGCTTTTTTGGCGGAACACACGGCCCCTCGCGCATTGGCCCGCGGGTCGCTCAAT
GAACCCGCATTGGTCCCCTGGGGTTCCGGGTATGGTAATGAGTTTCTTCGGGAAGGCGGGAAGCCCCG
GGGCACCGACGCAGGCCAAGCCCCTGTTGCGTCGGCGGGAGGGGCATGCTAATGGGGTTCTTTGGGGG
ACACCGGGTTGGGCCCCCAAATCGGGGGCCGGGCCGTGCATGCTAATGATATTCTTTGGGGGCGCCGG
GTTGGTCCCCGGGGACGGGGCCGCCCCGCGGTGGGCCTGCCTCCCCTGGGACGCGCGGCCATTGGGGG
AATCGTCACTGCCGCCCCTTTGGGGAGGGGAAAGGCGTGGGGTATAAGTTAGCCCTGGCCCGACAGTC
TGGTCGCATTTGCACCTCGGCACTCGGAGCGAGACGCAGCAGCCAGGCAGACTCGGGCCGCCCCCTCT
CCGCATCACCACAGAAGCCCCGCCTACGTTGCGACCCCCAGGGACCCTCCGTCCGCGACCCTCCAGCC
GCATACGACCCCC
RS1 promoter
[SEQ ID NO.: 3]
CCCGGGCCCCGCCCCCTGCCCGTTCCTCGTTAGCATGCGGAACGGAAGCGGAAACCGCCGGATCGGGC
GGTAATGAGATGCCATGCGGGGCGGGGCGCGGACCCACCCGCCCTCGCGCCCCGCCCATGGCAGATGG
CGCGGATGGGCGGGGCCGGGGGTTCGACCAACGGGCCGCGGCCACGGGCCCCCGGCGTGCCGGCGTCG
GGGCGGGGTCGTGCATAATGGAATTCCGTTCGGGGTGGGCCCGCCGGGGGGGCGGGGGGCCGGCGGCC
TCCGCTGCTCCTCCTTCCCGCCGGCCCCTGGGACTATATGAGCCCGAGGACGCCCCGATCGTCCACAC
GGAGCGCGGCTGCCGACACGGATCCACGACCCGACGCGGGACCGCCAGAGACAGACCGTCAGACGCTC
GCCGCGCCGGGACGCCGATACGCGGACGAAGCGCGGGAGGGGGATCGGCCGTCCCTGTCCTTTTTCCC
ACCCAAGCATCGACCGGTCCGCGCTAGTTCCGCGTCGACGGCGGGGGTCGTCGGGGTCCGTGGGTCTC
GCCCCCTCCCCCCATCGAGAGTCCGTAGGTGACCTACCGTGCTACGTCCGCCGTCGCAGCCGTATCCC
CGGAGGATCGCCCCGCATCGGCG
UL23 promoter
[SEQ ID NO.: 4]
GGATCCAAATGAGTCTTCGGACCTCGCGGGGGCCGCTTAAGCGGTGGTTAGGGTTTGTCTGACGCGGG
GGGAGGGGGAAGGAACGAAACACTCTCATTCGGAGGCGGCTCGGGGTTTGGTCTTGGTGGCCACGGGC
ACGCAGAAGAGCGCCGCGATCCTCTTAAGCACCCCCCCGCCCTCCGTGGAGGCGGGGGTTTGGTCGGC
GGGTGGTAACTGGCGGGCCGCTGACTCGGGCGGGTCGCGCGCCCCAGAGTGTGACCTTTTCGGTCTGC
TCGCAGACCCCCGGGCGGCGCCGCCGCGGCGGCGACGGGCTCGCTGGGTCCTAGGCTCAATGGGGACC
GTATACGTGGACAGGCTCTGGAGCATCCGCACGACTGCGGTGATATTACCGGAGACCTTCTGCGGGAC
GAGCCGGGTCACGCGGCTGACGCGGAGCGTCCGTTGGGCGACAAACACCAGGACGGGGCACAGGTACA
CTATCTTGTCACCCGGAGGCGCGAGGGACTGCAGGAGCTTCAGGGAGTGGCGCAGCTGCTTCATCCCC
GTGGCCCGTTGCTCGCGTTTGCTGGCGGTGTCCCCGGAAGAAATATATTTGCATGTCTTTAGTTCTAT
GATGACACAAACCCCGCCCAGCGTCTTGTCATTGGCGAATTCGAACACGCAGATGCAGTCGGGGCGGC
GCGGTCCCAGGTCCACTTCGCATATTAAGGTGACGCGTGTGGCCTCGAATACCGAGCGACCCTGCAGC
GACCCGCTTAACAGCGTCAACAGCGTGCCGCAGATCTTGGTGGCGTGAAACTCCCGCACCTCTTCGGC
CAGCGCCTTGTAGAAGCGCGT
UL27 promoter
[SEQ ID NO.: 5]
TCAACGGGCCCCTCTTTGATCACTCCACCCACAGCTTCGCCCAGCCCCCCAACACCGCGCTGTATTAC
AGCGTCGAGAACGTGGGGCTCCTGCCGCACCTGAAGGAGGAGCTCGCCCGGTTCATCATGGGGGCGGG
GGGCTCGGGTGCTGATTGGGCCGTCAGCGAATTTCAGAGGTTTTACTGTTTTGACGGCATTTCCGGAA
TAACGCCCACTCAGCGCGCCGCCTGGCGATATATTCGCGAGCTGATTATCGCCACCACACTCTTTGCC
TCGGTCTACCGGTGCGGGGAGCTCGAGTTGCGCCGCCCGGACTGCAGCCGCCCGACCTCCGAAGGTCG
TTACCGTTACCCGCCCGGCGTATATCTCACGTACGACTCCGACTGTCCGCTGGTGGCCATCGTCGAGA
GCGCCCCCGACGGCTGTATCGGCCCCCGGTCGGTCGTGGTCTACGACCGAGACGTTTTCTCGATCCTC
TACTCGGTCCTCCAGCACCTCGCCCCCAGGCTACCTGACGGGGGGCACGACGGGCCCCCGTAGTCCCG
CC
UL29 promoter
[SEQ ID NO.: 6]
CGGGCGGCGAGCTGCTGCGCGGCGCCCCGGCCGGCGGCCCGGTTTATTCGCGTCGGCCCGGCCGGCCG
GGCTTATGGACCGCCGGCGGCCGACAGGAGAGTGACGTAGCCGGTGGGCGTGGAGGGGCTGGGGCGGA
CCGGCACGCCCCCAGGTAAAGTGTACATATACCAACCGCATACCAGACGCACCCGACCCGGAGCACCT
GACCGTAAGCATCTGTGCCTCTCGCAGGGACCCCGCGTTGCCGGCCGCCGGGGTTCATCGGCACCCCG
TGGTTACCCGGGGGGTTGTCGGTGAAGGGGAGGGATTCATTCCCCAACCCCGGTCTCCAACCCTCCCC
TTGACCGTCGCCGCCCCCCCCCGGATTTTGACGCTCGGGAGACATACCTTGTCGGGCGTCCGTCGTCG
TGCCGGGATTACCTCCGTTCGCGGACCGATTGACAAAAGGAC
UL37 promoter
[SEQ ID NO.: 7]
CCCGGGCCTGGGTCCGCGAACGGGATGCCGGGACTTAAGTGGCCGTATAACACCCCGCGAAGACGCGG
GGTACTCGCAACGCCTGCGGGGGTCCTGGAGGGCCGCGGGGGATCGATAATTCGCCGCTCCCTACAGC
GCACGACAGTCATTCCCGCCCGGTCTCGTCGTTGGTCTACGCTGTCCCCCACCCACGCGAGCCGGGCG
TC
UL38 promoter
[SEQ ID NO.: 8]
CCCGGGGGATTGTCCGGATGTGCGGGCAGCCCGGACGGCGTGGGTTGCGGACTTTCTGCGGGGCGGCC
CAAATGGCCCTTTAAACGTGTGTATACGGACGCGCCGGGCCAGTCGGCCAACACAACCCACCGGAGGC
GGTAGCCGCGTTTGGCTGTGGGGTGGGTGGTTCCGCCTTGCGTGAGTGTCCTTTCGACCCCCCCCCCC
CTCCCTCCCCCGGGTCTTGCTAGGTCGCGATCTGGGGTCGCA
UL39 promoter
[SEQ ID NO.: 9]
CCGCTGTCACTCGTTGTTCGTTGACCCGGGCGTCCGCCAAATAAAGCCACTGAAACCCGAAACGCGAG
TGTTGTAACGTCCTTTGGGCGGGAGGAAGCCACAAAATGCAAATGGGATACATGGAAGGAACACACCC
CCGTGACTCAGGACATCGGTGTGTCCTTTTGGGTTTCACTGAAACTGGCCCGCGCCCCACCCCTGCGC
GATGTGGATAAAAAGCCAGCGCGGGTGGTTTAGGGTACCACAGGTGGGTGCTTTGGAAACTTGCCGGT
CGCCGTGCTCCTGTGAGCTTGCGTCCCTCCCCGGTTTCCTTTGCGCTCCCGCCTTCCGGACCTGCTCT
CGCCTATCTTCTTTGGCTCTCGGTGCGATTCGTCAGGCAGCGGCCTTGTCGAATCTCGACCCCACCAC
TCGCCGGACCCGCCGACGTCCCCTCTCGAGCCCGCCGAAACCCGCCGCGTCTGTTGAA
UL42 promoter
[SEQ ID NO.: 10]
ATTTCGATGGCCCAACTCCACGCGGATTGGTGCAGCACCCTGCATGCGCCGGTGCGGGCCAACCTTCC
CCCCGCTCATTGCCTCTTCCAAAAGGGTGTGGCCTAACGAGCTGGGGGCGTATTTAATCAGGCTAGCG
CGGCGGGCCTGCCGTAGTTTCTGGCTCGGTGAGCGACGGTCCGGTTGCTTGGGTCCCCTGGCTGCCAT
CAAAACCCCACCCTCGCAGCGGCATACGCCCCCTCCGCGTCCCGCACCCGAGACCCCGGCCCGGCTGC
CCTCACCACCGAAGCCCACCTCGTCACTGTGGGGTGTTCCCAGCCCGCGTTGGG
UL44 promoter
[SEQ ID NO.: 11]
GCAGGTCATCAACCTCGGGTTGGTGTTTCGGTTTTCCGAGGTTGTCGTGTATGCGGCGCTAGGGGGTG
CCGTGTGGATTTCGTTGGCGCAGGTGCTGGGGCTCCGGCGTCGCCTGCACAGGAAGGACCCCGGGGAC
GGGGCCCGGTTGGCGGCGACGCTTCGGGGCCTCTTCTTCTCCGTGTACGCGCTGGGGTTTGGGGTGGG
GGTGCTGCTGTGCCCTCCGGGGTCAACGGGCGGGCGGTCGGGCGATTGATATATTTTTCAATAAAAGG
CATTAGTCCCGAAGACCGCCGGTGTGTGATGATTTCGCCATAACACCCAAACCCCGGATGGGGCCCGG
GTATAAATTCCGGAAGGGGACACGGGCTACCCTCACTATCGAGGGCGCTTGGTCGGGAGGCCGCATCG
AACGCACACCCCCATCCGGTGGTCCGTGTGGAGGTCGTTTTCAGTGCCCGGTCTCGCTTTGCCGGGAA
CGCTAGCCGATCCCTCGCGAGGGGGAGGCGTCGGGC
UL48 promoter
[SEQ ID NO.: 12]
GGGGTTCATTCGGTGTTGGCGTTGCGTGCCTTTGTTTCCCAATCCGACGGGGACCGGGACTGGGTGGC
GGGGGGTGGGTTGGACAGCCGCCCTCGGTTCGCCTTCACGTGACAGGAGCCAATGTGGGGGGAAGTCA
CGAGGTACGGGGCGGCCCGTGCGGGTTGCTTAAATGCGTGGTGGCGACCACGGGCTGTCATTCCTCGG
GAACGGACGGGGTTCCCGCTGCCCACTTCCCCCCATAAGGTCCGTCCGGTCCTCTAACGCGTTTGGGG
GTTTTCTCTTCCCGCGCCGTCGGGCGTCCCACACTCTCTGGGCGGGCGGGGACGATCGCATCAAAAGC
CCGATATCGTCTTTCCCGTATCAACCCCACCCA
UL54 promoter
[SEQ ID NO.: 13]
ACCCCGCCCATGGGTCCCAATTGGCCGTCCCGTTACCAAGACCAACCCAGCCAGCGTATCCACCCCCG
CCCGGGTCCCCGCGGAAGCGGAACGGGGTATGTGATATGCTAATTAAATACATGCCACGTACTTATGG
TGTCTGATTGGTCCTTGTCTGTGCCGGAGGTGGGGCGGGGGCCCCGCCCGGGGGGCGGAACGAGGAGG
GGTTTGGGAGAGCCGGCCCCGGCACCACGGGTATAAGGACATCCACCACCCGGCCGGTGGTGGTGTGC
AGCCGTGTTCCAACCACGGTCACGCTTCGGTGCCTCTCCCCGATTCGGGCCCGGTCGCTCGCTACCGG
TGCGCCACCACCAGAGGCCATATCCGACACCCCAGCCCCGACGGCAGCCGACAGCCCGGTC
US6 promoter
[SEQ ID NO.: 14]
CTGCTTGAGCTCCTGCGTCGTACGTGCCGCGGGTGGGGGCGTTACCATCCCTACATGGACCCAGTTGT
CGTATAATTTCCCCCCCCCCCCCCCTTCTCCGCGTGGGTGATGTCGGGTCCAAACTCCCGACACCACC
AGCTGGCATGGTATAAATCACCGGTGCGCCCCCCAAACCATGTCCGGCAGGGGGATGGGGGGGCGAAT
GCGGAGGGCACCCAACAACACCGGGCTAACCAGGAAATCCGTGGCCCCGGCCCCCAATAAAGATCGCG
GTAGCCCGGCCGTGTGACACTATCGTCCATACCGACCACACCGACGAATCCCCCAAGGGGGAGGGGCC
ATTTTACGAGGAGGAGGGGTATAACAAAGTCTGTCTTTAAAAAGCAGGGGTTAGGGAGTTGTTCGGTC
ATAAGCTTCAGCGCGAACGACCAACTACCCCGATCATCAGTTATCCTTAAGGTCTCTTTTGTGTGGTG
CGTTCCGGT

“Essential viral gene” refers to a viral gene that is essential in certain but not necessarily all circumstances for the survival, replication, and/or propagation of the virus in vivo. “Essential HSV-1 gene” refers to a HSV-1 gene that is essential in certain but not all circumstances for the survival replication, and/or propagation of HSV-1 virus in vivo. Non-limiting examples of essential HSV-1 genes include RL2 gene, RS1 gene, UL54 gene, US1 gene, US1.5 gene, US12 gene, UL5 gene, UL8 gene, UL9 gene, UL23 gene, UL29 gene, UL30 gene, UL42 gene, UL52 gene, UL1 gene, UL6 gene, UL15 gene, UL16 gene, UL18 gene, UL19 gene, UL22 gene, UL26 gene, UL26.5 gene, UL27 gene, UL28 gene, UL31 gene, UL32 gene, UL33 gene, UL34 gene, UL35 gene, UL36 gene, UL37 gene, UL38 gene, UL48 gene, UL49.5 gene, and US6 gene.

Non-limiting examples of viral genes include immediate-early viral genes (or “IE gene”), early viral genes (or “E gene”), and late viral genes (or “L gene”). “Immediate-early gene” or “IE gene” or “α gene” refers to genes that are activated and transcribed immediately after viral infection, in the absence of de novo protein synthesis. The IE proteins encoded by the corresponding IE genes are responsible for regulating viral gene expression during subsequent phases of the replication cycle (Sanfilippo et al., Journal of Virology (2018); 92(2)224-39). The IE genes act in part to up-regulate the expression of the early genes. Non-limiting examples of immediate-early genes of HSV-1 include RL2 gene, RS1 gene, UL54 gene, US1 gene, US1.5 gene, and US12 gene. In certain embodiments, the immediate-early genes are selected from the group consisting of a RL2 gene, a RS1 gene, and a UL54 gene.

“Early gene” or “E gene” or “β gene” refers to genes that encode proteins required for viral DNA synthesis. The expression of early genes is regulated by the IE proteins (Pesola et al., Journal of Virology (2005); 79(23):14516-25). In HSV-1, the function of several early genes is to turn off the expression of the immediate-early gene and to induce the expression of the late genes. Non-limiting examples of early genes of HSV-1 include, but not limited to UL5 gene, UL8 gene, UL9 gene, UL23 gene, UL29 gene, UL30 gene, UL42 gene, and UL52 gene. In certain embodiments, the early gene is a UL29 gene.

“Late gene” or “L gene” or “y gene” refers to genes that are required for DNA replication for maximal expression. Late genes mainly encode structural proteins, and start to be transcribed following viral DNA replication. The expression of late genes ultimately leads to the assembly and release of infectious particles (Gruffat, Frontiers in Microbiology (2016); 7:869). Non-limiting examples of late genes of HSV-1 include UL1 gene, UL6 gene, UL15 gene, UL16 gene, UL18 gene, UL19 gene, UL22 gene, UL26 gene, UL26.5 gene, UL27 gene, UL28 gene, UL31 gene, UL32 gene, UL33 gene, UL34 gene, UL35 gene, UL36 gene, UL37 gene, UL38 gene, UL48 gene, UL49.5 gene, and US6 gene. In certain embodiments, the late genes are selected from the group consisting of a UL6 gene, a UL15 gene, a UL19 gene, a UL22 gene, a UL32 gene, a UL33 gene, a UL37 gene, and a UL48 gene.

In various non-limiting embodiments, the genome editing systems of the present disclosure target two or more (e.g., three, four, or five) specific nucleotide sequences through the use of a combination of two or more (e.g., three, four, or five) gRNAs. In certain embodiments, the genome editing systems of the present disclosure target two specific nucleotide sequences through the use of a combination of two gRNAs. In certain embodiments, the genome editing systems of the present disclosure target three specific nucleotide sequences through the use of a combination of three gRNAs. In certain embodiments, the genome editing systems of the present disclosure target four specific nucleotide sequences through the use of a combination of four gRNAs. In certain embodiments, the genome editing systems of the present disclosure target five specific nucleotide sequences through the use of a combination of five gRNAs.

In certain embodiments, a cell is manipulated by editing (e.g., introducing a mutation in) one or more target viral genes. In certain embodiments, the expression of one or more target genes are modulated, e.g., in vivo.

In certain embodiments, the method comprises delivery of gRNA by an AAV.

Non-limiting exemplary AAV vectors include serotype 1, 2, 3, 4, 5, 6, 7, 8 or 9 vectors. In certain embodiments, the method comprises delivery of gRNA by a lentivirus. In certain embodiments, the method comprises delivery of gRNA by a nanoparticle. In certain embodiments, the method comprises delivery of gRNA by a gel-based AAV for topical therapy.

Methods to Treat, Prevent, and/or Reduce Viral Infection and Viral Infection-Related Diseases

Disclosed herein are methods to treat, prevent, and/or reduce viral infections and viral infection-related diseases using the gene editing systems disclosed herein, where the expression of at least one component of the gene editing system is regulated by a promoter derived from the targeted viral family, genus, and/or species.

In certain embodiments, the viral infection or viral infection-related disease is a Herpesviridae family virus infection or virus infection-related disease. In certain embodiments, the viral infection or viral infection-related disease is a Alphaherpesvirinae subfamily virus infection or virus infection-related disease, a Betaherpesvirinae subfamily virus infection or virus infection-related disease, or a Gammaherpesvirinae subfamily virus infection or virus infection-related disease. In certain embodiments, the virus infection or virus infection-related disease is an Iltovirus infection or infection-related disease, a Mardivirus infection or infection-related disease, a Simplexvirus infection or infection-related disease, a Scutavirus infection or infection-related disease, or a Varicellovirus virus infection or virus infection-related disease. In certain embodiments, the virus infection or virus infection-related disease is a HSV, HSV-1, or HSV-2 infection or infection-related disease. In certain embodiments, the virus infection or virus infection-related disease is a Cytomegalovirus (CMV) infection or infection-related disease, a Morumegalovirus infection or infection-related disease, a Proboscivirus infection or infection-related disease, or a Roseolovirus infection or infection-related disease. In certain embodiments, the virus infection or virus infection-related disease is a Lymphocryptovirus infection or infection-related disease, a Macavirus infection or infection-related disease, a Percavirus infection or infection-related disease, or a Rhadinovirus infection or infection-related disease. In certain embodiments, the virus infection or virus infection-related disease is a human herpesvirus infection or infection-related disease, such as a Human Cytomegalovirus (HCMV) infection or infection-related disease, a Kaposi Sarcoma-Associated Herpesvirus (KSHV) infection or infection-related disease, an Epstein-Barr virus (EBV) infection or infection-related disease, and a Varicella-Zoster virus (VZV) infection or infection-related disease. In certain embodiments, the infection or infection-related disease is a Human Immunodeficiency Virus (HIV) infection or infection-related disease or a Human Papillomavirus (HPV) infection or infection-related disease.

In certain embodiments, the viral infection is a CMV infection. In certain embodiments, the viral infection-related-disease is a CMV-related disease.

In certain embodiments, the viral infection is an HSV infection. In certain embodiments, the viral infection is an HSV-1 infection, an HSV-2 infection, or an HSV-1 and HSV-2 infection. In certain embodiments, the viral infection-related disease is an HSV-related disease. In certain embodiments, the HSV-related disease is an HSV-related ocular disease. HSV-related ocular infections can be caused by an HSV-1 and/or an HSV-2 infection.

The methods, systems, vectors, and compositions disclosed herein can be used to treat, prevent, and/or reduce an HSV-1 infection, and/or an HSV-2 infection. In certain embodiments, the HSV-related ocular keratitis is a recurrent ocular keratitis, including but not limited to, HSV-1 recurrent ocular keratitis and/or HSV-2 recurrent ocular keratitis. In certain embodiments, the genome editing systems, compositions and methods described herein can be used for the treatment, prevention and/or reduction of HSV-1 and/or HSV-2 ocular infections, including but not limited to HSV-1 stromal keratitis, HSV-1 dendritic keratitis, HSV-1 blepharitis, HSV-1 conjunctivitis, HSV-1 retinitis, HSV-2 stromal keratitis, HSV-2 dendritic keratitis, HSV-2 blepharitis, HSV-2 conjunctivitis, and HSV-2 retinitis.

In certain embodiments, inhibiting essential viral functions, e.g., viral gene transcription, viral genome replication and viral capsid formation, decreases the duration of recurrent infection and/or decrease shedding of viral particles. In certain embodiments, subjects also experience shorter duration(s) of illness, decreased risk of transmission to sexual partners, decreased risk of transmission to the fetus in the case of pregnancy and/or the potential for full clearance of virus (e.g., HSV-1, HSV-2, and/or CMV) (cure).

Knockout of one or more copies (e.g., 1, 2, 3, 4, 5, 6, 7, 8, 9 or 10 copies) of one or more viral genes can be performed prior to disease onset or after disease onset (including early in the disease course).

In certain embodiments, the method comprises initiating treatment of a subject prior to disease onset. In certain embodiments, the method comprises initiating treatment of a subject after disease onset. In certain embodiments, the method comprises initiating treatment of a subject well after disease onset, e.g., 1, 2, 3, 4, 5, 6, 7, 8, 9, 10, 12, 16, 24, 36, 48 or more months after onset of viral infection (e.g., HSV-1, HSV-2, and/or CMV infections). In certain embodiments, the method comprises initiating treatment of a subject well after disease onset, e.g., 1, 2, 3, 4, 5, 10, 15, 20, 25, 40, 50 or 60 years after onset of viral infection. This can be effective as disease progression is slow in some cases and a subject can present well into the course of illness.

In certain embodiments, the method comprises initiating treatment of a subject in an advanced stage of disease, e.g., during latent periods. In certain embodiments, the method comprises initiating treatment of a subject in the case of severe, acute disease affecting eyes. Overall, initiation of treatment for subjects at all stages of disease is expected to improve healing, decrease duration of disease and be of benefit to subjects.

In certain embodiments, the method comprises initiating treatment of a subject prior to disease progression. In certain embodiments, the method comprises initiating treatment of a subject in an early stage of disease, e.g., when a subject has been exposed to a virus (e.g., HSV-1, HSV-2, and/or CMV) or is thought to have been exposed to the virus (e.g., HSV-1, HSV-2, and/or CMV). In certain embodiments, the method comprises initiating treatment of a subject prior to disease expression. In certain embodiments, the method comprises initiating treatment of a subject in an early stage of disease, e.g., when a subject has been tested positive for the virus infections (e.g., HSV-1, HSV-2, and/or CMV infections) but has no signs or symptoms.

In certain embodiments, the method comprises initiating treatment at the appearance of one or more of the following findings consistent or associated with a viral infection (e.g., HSV-1, HSV-2, and/or CMV infections): fever, headache, body aches, ano-genital blistering, oral ulceration, encephalitis, or keratitis. In certain embodiments, the method comprises initiating treatment of a subject at the appearance of painful blistering in or around the mouth, e.g., oral or oropharynx, e.g., in an infant, child, adult or young adult. In certain embodiments, the method comprises initiating treatment of a subject at the appearance of painful blistering in the ano-genital region, genital ulcers, and/or a flu-like symptom, e.g., in an infant, child, adult or young adult.

In certain embodiments, the method comprises initiating treatment of a subject suspected of having viral-related (e.g., HSV-1, HSV-2, and/or CMV) meningitis and/or viral-related (e.g., HSV-1, HSV-2, and/or CMV) encephalitis. In certain embodiments, the method comprises initiating treatment at the appearance of one or more of the following symptoms consistent or associated with viral-related (e.g., HSV-1, HSV-2, and/or CMV) meningitis and/or encephalitis: fever, headache, vomiting, photophobia, seizure, decline in level of consciousness, lethargy, or drowsiness.

In certain embodiments, the method comprises initiating treatment at the appearance of any of the following signs consistent or associated with viral-related (e.g., HSV-1, HSV-2, and/or CMV) meningitis and/or encephalitis: positive CSF culture for the virus, elevated WBC in CSF, neck stiffness/positive Brudzinski's sign. In certain embodiments, the method comprises initiating treatment in a patient with signs consistent with viral-related (e.g., HSV-1, HSV-2, and/or CMV) encephalitis and/or meningitis on EEG, CSF exam, MRI, PCR of CSF specimen, and/or PCR of brain biopsy specimen.

In certain embodiments, the method comprises initiating treatment at the appearance of any of the following symptoms consistent or associated with optic viral disease (e.g., HSV-1, HSV-2, and/or CMV infections): pain, photophobia, blurred vision, tearing, redness/injection, loss of vision, floaters, or flashes.

In certain embodiments, the method comprises initiating treatment at the appearance of any of the following findings on ophthalmologic exam consistent or associated with optic viral disease (e.g., HSV-1, HSV-2, and/or CMV infections), also known as viral related keratitis (HSV-1, HSV-2, and/or CMV related keratitis): small, raised clear vesicles on corneal epithelium; irregular corneal surface, punctate epithelial erosions; dense stromal infiltrate; ulceration; necrosis; focal, multifocal, or diffuse cellular infiltrates; immune rings; neovascularization; or ghost vessels at any level of the cornea.

In certain embodiments, the method comprises initiating treatment at the appearance of any of the following findings on ophthalmologic exam consistent or associated with viral related (e.g., HSV-1, HSV-2, and/or CMV related) retinitis or acute retinal necrosis: reduced visual acuity; uveitis; vitritis; scleral injection; inflammation of the anterior and/or vitreous chamber/s; vitreous haze; optic nerve edema; peripheral retinal whitening; retinal tear; retinal detachment; retinal necrosis; evidence of occlusive vasculopathy with arterial involvement, including arteriolar sheathing and arteriolar attenuation.

In certain embodiments, the method comprises initiating treatment at the appearance of symptoms and/or signs consistent or associated with either a viral infection (e.g., HSV-1, HSV-2, and/or CMV infections) of the eye, oropharynx, ano-genital region or central nervous system. In certain embodiments, initiating treatment for a viral infection (e.g., HSV-1, HSV-2, and/or CMV infections) in a case of suspected the viral infection (e.g., HSV-1, HSV-2, and/or CMV infections) early in the disease course is beneficial.

In certain embodiments, the method comprises initiating treatment in utero. In certain embodiments, the subject is at high risk of maternal-to-fetal transmission.

In certain embodiments, the method comprises initiating treatment during pregnancy in case of mother who has an active viral infection (e.g., HSV-1, HSV-2, and/or CMV infections) or has recent primary viral infection (e.g., HSV-1, HSV-2, and/or CMV infections).

In certain embodiments, the method comprises initiating treatment prior to organ transplantation or immediately following organ transplantation.

In certain embodiments, the method comprises initiating treatment in case of suspected exposure to a virus (e.g., HSV-1, HSV-2, and/or CMV).

In certain embodiments, the method comprises initiating treatment prophylactically, in case of suspected a viral related (e.g., HSV-1, HSV-2, and/or CMV related) encephalitis or meningitis.

In certain embodiments, the method comprises initiating treatment prior to organ transplantation or immediately following organ transplantation.

In certain embodiments, the method comprises initiating treatment in case of suspected exposure to a virus (e.g., HSV-1, HSV-2, and/or CMV).

In certain embodiments, the method comprises initiating treatment of a subject who suffers from or is at risk of developing severe manifestations of a viral infection, e.g., neonates, subjects with HIV, subjects who are on immunosuppressant therapy following organ transplantation, subjects who have cancer, subjects who are undergoing chemotherapy, subjects who will undergo chemotherapy, subjects who are undergoing radiation therapy, subjects who will undergo radiation therapy.

Both HIV positive subjects and post-transplant subjects can experience severe viral (e.g., HSV-1 and CMV) activation or reactivation due to immunodeficiency. Neonates are also at risk for severe viral-related encephalitis due to maternal-fetal transmission during childbirth. Inhibiting essential viral functions, e.g., viral gene transcription, viral genome replication and viral capsid formation, can provide superior protection to said populations at risk for severe viral infections. Subjects can experience lower rates of viral encephalitis and/or lower rates of severe neurologic sequelae following viral encephalitis, which will profoundly improve quality of life.

In certain embodiments, the method comprises initiating treatment in any subject who has been exposed to viral and at high risk for severe viral infection (e.g., HSV-1, HSV-2, and/or CMV infections).

### Genome Editing Systems

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nanoparticle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”, incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include at least one component that promotes or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated by reference. Alternatively, a genome editing system may utilize a cleavage-inactivated (i.e. a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. In certain embodiments, this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (Nishimasu 2014) and Nishimasu et al., Cell 162, 1113-1126, Aug. 27, 2015 (Nishimasu 2015), both incorporated by reference herein). It should be noted that the first and/or second complementarity domains may contain one or more poly-A tracts, which can be recognized by RNA polymerases as a termination signal. The sequence of the first and second complentarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate that, although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

gRNA Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. Each of these references is incorporated by reference herein. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. In certain embodiments, the modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5)ppp(5)G cap analog, a m7G(5)ppp(5)G cap analog, or a 3′-O-Me-m7G(5)ppp(5)G anti reverse cap analog (ARCA)), as shown below:

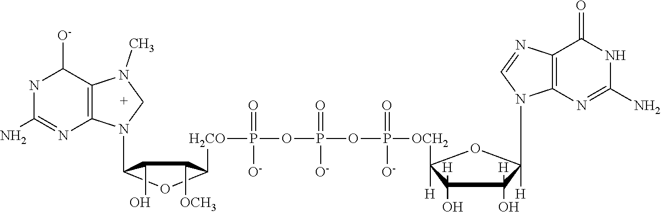

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g., a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

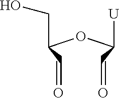

wherein “U” can be an unmodified or modified uridine.

The 3′ terminal U ribose can be modified with a 2′3′ cyclic phosphate as shown below:

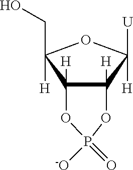

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, 0- and N-alkylated nucleotides, e.g., N6-methyl adenosine, can be incorporated into the gRNA. In certain embodiments, one or more or all of the nucleotides in a gRNA are deoxynucleotides.

Non-limiting exemplary strategies, methods, and compositions suitable for editing a target nucleic acid sequence, or modulating expression of a target nucleic acid sequence, e.g., a target nucleic acid sequence of a gene or genome of a virus, e.g., an HSV, an HSV-1, an HSV-2, or a CMV, have been disclosed herein. Based on the instant disclosure, additional suitable strategies, methods, and compositions will be apparent to those of skill in the art. Any suitable gRNAs or gRNAs known in the art can be used with the presently disclosed subject matter. It will be understood, for example, and without limitation, that guide RNAs other than those known in the art can be used for editing a target nucleic acid sequence, or modulating expression of a target nucleic acid sequence, e.g., a target nucleic acid sequence of a gene or genome of a virus, e.g., an HSV, an HSV-1, an HSV-2, or a CMV. Non-limiting exemplary methods for designing guide RNAs are disclosed herein and additional suitable methods will be apparent to the skilled artisan based on the present disclosure and the knowledge in the art. In certain embodiments envisioned, a guide RNA is designed and/or used in a strategy, method, and/or compositions for editing or modulating expression of a target nucleic acid sequence of a virus gene or genome that binds to a target site within about 5 nucleotides, within about 10 nucleotides, within about 20 nucleotides, within about 25 nucleotides, within about 30 nucleotides, within about 40 nucleotides, within about 50 nucleotides, within about 60 nucleotides, within about 70 nucleotides, within about 75 nucleotides, within about 80 nucleotides, within about 90 nucleotides, within about 100 nucleotides, within about 200 nucleotides, within about 250 nucleotides, within about 300 nucleotides, within about 400 nucleotides, within about 500 nucleotides, within about 600 nucleotides, within about 700 nucleotides, within about 750 nucleotides, within about 800 nucleotides, within about 900 nucleotides, or within about 1000 nucleotides from the target site of any of the suitable guide RNAs or gRNAs known in the art that can be used with the presently disclosed subject matter.

In certain embodiments, a guide RNA is designed and/or used in a strategy, method, and/or compositions for editing or modulating expression of a target nucleic acid sequence of a virus gene or genome that binds to a target site within about 5 nucleotides, within about 10 nucleotides, within about 20 nucleotides, within about 25 nucleotides, within about 30 nucleotides, within about 40 nucleotides, within about 50 nucleotides, within about 60 nucleotides, within about 70 nucleotides, within about 75 nucleotides, within about 80 nucleotides, within about 90 nucleotides, within about 100 nucleotides, within about 200 nucleotides, within about 250 nucleotides, within about 300 nucleotides, within about 400 nucleotides, within about 500 nucleotides, within about 600 nucleotides, within about 700 nucleotides, within about 750 nucleotides, within about 800 nucleotides, within about 900 nucleotides, or within about 1000 nucleotides from the target site of any of the suitable guide RNAs or gRNAs known in the art to show efficacy in editing or modulating, e.g., reducing or abolishing, expression of a target nucleic acid sequence of a virus gene or genome.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that that do not cut at all.

Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. In certain embodiments, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a psuedonot structure, rather than a stem loop structure formed by the repeat:antirepeat duplex in Cas9 gRNAs.

Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g., Cotta-Ramusino. The stability of RNP complexes may be evaluated by differential scanning fluorimetry, as described below.

Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g., different stoichiometric ratios of gRNA: RNA-guided nuclease protein, different buffer solutions, etc.) to identify optimal conditions for RNP formation; and (b) testing modifications (e.g., chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g., 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g., 2 μM) of Cas9 in water+10×SYPRO Orange® (Life Technologies cat #S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g., 2 μM) Cas9 in optimal buffer from assay 1 above and incubating (e.g., at RT for 10′) in a 384 well plate. An equal volume of optimal buffer+10×SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g., SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g., a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g., a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g., ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

Multiplex Strategies

While exemplary strategies discussed above have focused on repair outcomes mediated by single DSBs, genome editing systems according to this disclosure may also be employed to generate two or more DSBs, either in the same locus or in different loci. Strategies for editing that involve the formation of multiple DSBs, or SSBs, are described in, for instance, Cotta-Ramusino.

Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g., flanking or adjoining) a target sequence to be cleaved. These homologous regions are referred to here as “homology arms,” and are illustrated schematically below:

[5′ homology arm]-[replacement sequence]→[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g., in an AAV or lentiviral genome) that includes certain genomic backbone elements (e.g., inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

### Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g., administered to a subject) immediately, or they can be maintained or stored for later use. Those of skill in the art will appreciate that cells can be maintained in culture or stored (e.g., frozen in liquid nitrogen) using any suitable method known in the art.

The compositions or systems described herein can be delivered to a target cell. In certain embodiments, the target cell is an epithelial cell, e.g., an epithelial cell of the oropharynx (including, e.g., an epithelial cell of the nose, gums, lips, tongue or pharynx), an epithelial cell of the finger or fingernail bed, or an epithelial cell of the ano-genital area (including, e.g., an epithelial cell of the penis, scrotum, vulva, vagina, cervix, anus or thighs). In certain embodiments, the target cell is a neuronal cell, e.g., a cranial ganglion neuron (e.g., a trigeminal ganglion neuron, e.g., an oculomotor nerve ganglion neuron, e.g., an abducens nerve ganglion neuron, e.g., a trochlear nerve ganglion neuron), e.g., a cervical ganglion neuron, e.g., a sacral ganglion neuron, a sensory ganglion neuron, a cortical neuron, a cerebellar neuron or a hippocampal neuron. In an embodiment, the target cell is an optic cell, e.g., an epithelial cell of the eye, e.g., an epithelial cell of the eyelid, e.g., a conjunctival cell, e.g., a conjunctival epithelial cell, e.g., a corneal keratocyte, e.g., a limbus cell, e.g., a corneal epithelial cell, e.g., a corneal stromal cell, e.g., a ciliary body cell, e.g., a scleral cell, e.g., a lens cell, e.g., a choroidal cell, e.g., a retinal cell, e.g., a rod photoreceptor cell, e.g., a cone photoreceptor cell, e.g., a retinal pigment epithelium cell, e.g., a horizontal cell, e.g., an amacrine cell, e.g., a ganglion cell.

In certain embodiments, the compositions or systems described herein can be delivered to one or more of the following cell types: Muller cells; Bipolar Cells; Ciliary muscle cells; Suspensory ligaments; Iris muscle cells; Bruch's membrane cells; Trabecular meshwork cells; and Zonule fibers, as well as any nerve cells that innervate the eye.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 5 and 6 set forth several, non-limiting examples of genome editing system implementations. Those of skill in the art will appreciate, however, that these listings are not comprehensive, and that other implementations are possible. With reference to Table 5 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 5
Genome Editing System Components

| RNA-guided | | Donor | |
|---|---|---|---|
| Nuclease | gRNA | Template | Comments |
| Protein | RNA | [N/A] | An RNA-guided nuclease protein |
| | | | complexed with a gRNA molecule |
| | | | (an RNP complex) |
| Protein | RNA | DNA | An RNP complex as described |
| | | | above plus a single-stranded or |
| | | | double stranded donor template. |
| Protein | DNA | [N/A] | An RNA-guided nuclease protein |
| | | | plus gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein |
| | | | and a single DNA encoding both a |
| | | | gRNA and a donor template. |
| DNA | | | A DNA or DNA vector encoding |
| | | | an RNA-guided nuclease, a gRNA |
| | | | and a donor template. |
| DNA | DNA | [N/A] | Two separate DNAs, or two |
| | | | separate DNA vectors, encoding |
| | | | the RNA-guided nuclease and the |
| | | | gRNA, respectively. |
| DNA | DNA | DNA | Three separate DNAs, or three |
| | | | separate DNA vectors, encoding |
| | | | the RNA-guided nuclease, the |
| | | | gRNA and the donor template, |
| | | | respectively. |
| DNA | | [N/A] | A DNA or DNA vector encoding |
| | | | an RNA-guided nuclease and a |
| | | | gRNA |
| DNA | | DNA | A first DNA or DNA vector |
| | | | encoding an RNA-guided nuclease |
| | | | and a gRNA, and a second DNA or |
| | | | DNA vector encoding a donor |
| | | | template. |
| DNA | DNA | | A first DNA or DNA vector |
| | | | encoding an RNA-guided nuclease |
| | | | and second DNA or DNA vector |
| | | | encoding a gRNA and a donor |
| | | | template. |
| DNA | | | A first DNA or DNA vector |
| | DNA | | encoding an RNA-guided nuclease |
| | | | and a donor template, and a second |
| | | | DNA or DNA vector encoding a |
| | | | gRNA. |
| DNA | | | A DNA or DNA vector encoding |
| | RNA | | an RNA-guided nuclease and a |
| | | | donor template, and a gRNA |

| | | |
|---|---|---|
| RNA | [N/A] | An RNA or RNA vector encoding |
| | | an RNA-guided nuclease and |
| | | comprising a gRNA |
| RNA | DNA | An RNA or RNA vector encoding |
| | | an RNA-guided nuclease and |
| | | comprising a gRNA, and a DNA or |
| | | DNA vector encoding a donor |
| | | template. |

Table 6 summarizes various delivery methods for the components of genome editing systems, as described herein. Again, the listing is intended to be exemplary rather than limiting.

TABLE 6

| | | Delivery into | Duration | | |
|---|---|---|---|---|---|
| | | Non-Dividing | of | Genome | Type of Molecule |
| Delivery Vector/Mode | | Cells | Expression | Integration | Delivered |
| Physical (e.g., | | YES | Transient | NO | Nucleic |
| electroporation, particle | | | | | Acids and |
| gun, Calcium Phosphate | | | | | Proteins |
| transfection, cell | | | | | |
| compression or squeezing) | | | | | |
| Viral | Retrovirus | NO | Stable | YES | RNA |
| | Lentivirus | YES | Stable | YES/NO | RNA |
| | | | | with | |
| | | | | modifications | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes | YES | Stable | NO | DNA |
| | Simplex Virus | | | | |
| Non-Viral | Cationic | YES | Transient | Depends on | Nucleic |
| | Liposomes | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated | YES | Transient | NO | Nucleic |
| Non-Viral | Bacteria | | | | Acids |
| Delivery | Engineered | YES | Transient | NO | Nucleic |
| Vehicles | Bacteriophages | | | | Acids |
| | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector-based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 6, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 6, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g., lipid and/or polymer) nonparticles can be suitable for use as delivery vehicles in certain embodiments of this disclosure. Exemplary lipids for use in nanoparticle formulations, and/or gene transfer are shown in Table 7, and Table 8 lists exemplary polymers for use in gene transfer and/or nanoparticle formulations.

TABLE 7
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3- | DOPE | Helper |
| phosphatidylethanolamine | | |
| Cholesterol | | Helper |
| N-[1-(2,3-Dioleyloxy)propyl]N,N,N- | DOTMA | Cationic |
| trimethylammonium chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3 | GAP-DLRIE | Cationic |
| bis(dodecyloxy)-1-propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6- | 2Oc | Cationic |
| trimethylpyridinium | | |
| 2,3-Dioleyloxy-N-[2(sperminecarboxamido-ethyl]- | DOSPA | Cationic |
| N,N-dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl- | CCS | Cationic |
| spermine | | |
| N-t-Butyl-N0-tetradecyl-3- | diC14-amidine | Cationic |
| tetradecylaminopropionamidine | | |
| Octadecenolyoxy[ethyl-2-heptadecenyl-3 | DOTIM | Cationic |
| hydroxyethyl] imidazolinium chloride | | |
| N1-Cholesteryloxycarbonyl-3,7-diazanonane-1,9- | CDAN | Cationic |
| diamine | | |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]- | DLin-KC2- | Cationic |
| dioxolane | DMA | |
| dilinoleyl-methyl-4-dimethylaminobutyrate | DLin-MC3- | Cationic |
| | DMA | |

TABLE 8
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo.

In vitro, delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically, the microspheres are composed of a polymer of lactic acid and glycolic acid, which are structured to form hollow spheres. The spheres can be approximately 15-30 microns in diameter and can be loaded with components described herein.

Multi-Modal or Differential Delivery of Components

Skilled artisans will appreciate, in view of the instant disclosure, that different components of genome editing systems disclosed herein can be delivered together or separately and simultaneously or nonsimultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by WIC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only being formed in the tissue that is targeted by both vectors.

### EXEMPLARY EMBODIMENTS

A. In certain non-limiting embodiments, the presently disclosed subject matter provides a genome editing system comprising: (a) an RNA-guided nuclease, and (b) a gRNA molecule comprising a targeting domain that is complementary with a target sequence of a target gene of a targeted virus; wherein the expression of (a) and/or (b) is regulated by a promoter that is derived from a genome of the family, genus, or species of the targeted virus.

A1. The foregoing genome editing system of A, wherein the promoter is derived from a gene of the family, genus, or species of the targeted virus.

A2. The foregoing genome editing system of A or A1, wherein the promoter is operably linked to a polynucleotide encoding (a), and/or a polynucleotide encoding (b).

A3. The foregoing genome editing system of any one of A-A2, wherein the promoter is derived from an immediate early gene, an early gene, or a late gene of the family, genus, or species of the targeted virus.

A4. The foregoing genome editing system of any one of A-A3, wherein the expression of (a) and/or (b) is weak during a viral latency.

A5. The foregoing genome editing system of any one of A-A4, wherein the expression of (a) and/or (b) is strong during a viral reactivation.

A6. The foregoing genome editing system of A5, wherein the expression of (a) and/or (b) during the viral latency is at a level at least about 10%, at least about 20%, at least about 30%, at least about 40% or at least about 50% lower than the expression of (a) and/or (b) during the viral reactivation.

A7. The foregoing genome editing system of any one of A-A6, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Epstein-Barr virus, and a Kaposi's sarcoma-associated herpesvirus.

A8. The foregoing genome editing system of any one of A-A7, wherein the targeted virus is selected from the group consisting of a Simplexvirus, a Varicellovirus, a Cytomegalovirus, a Roseolovirus, a Lymphocryptovirus, and a Rhadinovirus.

A9. The foregoing genome editing system of any one of A-A8, wherein the targeted virus is a Herpes Simplex Virus (HSV).

A10. The foregoing genome editing system of any one of A-A9, wherein the targeted virus is a Herpes Simplex Virus-1 (HSV-1).

A11. The foregoing genome editing system of any one of A-A10, wherein the RNA-guided nuclease is a Cas9 molecule.

A12. The foregoing genome editing system of A11, wherein the Cas9 molecule comprises an S. pyogenes Cas9 molecule or an S. aureus Cas9 molecule.

A13. The foregoing genome editing system of claim A11 or A12, wherein the Cas9 molecule comprises a wild-type Cas9 molecule, a mutant Cas9 molecule, or a combination thereof.

A14. The foregoing genome editing system of A13, wherein the mutant Cas9 molecule comprises a D10A mutation.

A15. The foregoing genome editing system of any one of A-A14, wherein the RNA-guided nuclease is a Cpf1 molecule.

A16. The foregoing genome editing system of any one of A-A15, wherein the promoter is activated by a transactivator of a genome of the family, genus, or species of the targeted virus.

A17. The foregoing genome editing system of A16, wherein the transactivator is selected from a group consisting of a HSV-1 ICP0 protein, a HSV-1 ICP4 protein, and a HSV-1 ICP27 protein.

B. In certain non-limiting embodiments, the presently disclosed subject matter provides a composition comprising: (a) an RNA-guided nuclease, and (b) a gRNA molecule comprising a targeting domain that is complementary with a target sequence of a target gene of a targeted virus; wherein the expression of (a) and/or (b) is regulated by a promoter that is derived from a genome of the family, genus, or species of the targeted virus.

B1. The foregoing composition of B, wherein the promoter is derived from a gene of the family, genus, or species of the targeted virus.

B2. The foregoing composition of B or B1, wherein the promoter is operably linked to a polynucleotide encoding (a), and/or a polynucleotide encoding (b).

B3. The foregoing composition of any one of B-B2, wherein the promoter is derived from an immediate early gene, an early gene, or a late gene of the family, genus, or species of the targeted virus.

B4. The foregoing composition of any one of B-B3, wherein the expression of (a) and/or (b) is weak during a viral latency.

B5. The foregoing composition of any one of B-B4, wherein the expression of (a) and/or (b) is strong during a viral reactivation.

B6. The foregoing composition of B5, wherein the expression of (a) and/or (b) during the viral latency is at a level at least about 10%, at least about 20%, at least about 30%, at least about 40% or at least about 50% lower than the expression of (a) and/or (b) during the viral reactivation.

B7. The foregoing composition of any one of B-B6, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Eppstein-Barr virus, and a Kaposi's sarcoma-associated herpesvirus.

B8. The foregoing composition of any one of B-B7, wherein the targeted virus is selected from the group consisting of a Simplexvirus, a Varicellovirus, a Cytomegalovirus, a Roseolovirus, a Lymphocryptovirus, and a Rhadinovirus.

B9. The foregoing composition of any one of B-B8, wherein the targeted virus is a Herpes Simplex Virus (HSV).

B10. The foregoing composition of any one of B-B9, wherein the targeted virus is a Herpes Simplex Virus-1 (HSV-1).

B11. The foregoing composition of any one of B-B10, wherein the RNA-guided nuclease is a Cas9 molecule.

B12. The foregoing composition of B11, wherein the Cas9 molecule comprises an S. pyogenes Cas9 molecule or an S. aureus Cas9 molecule.

B13. The foregoing composition of B11 or B12, wherein the Cas9 molecule comprises a wild-type Cas9 molecule, a mutant Cas9 molecule, or a combination thereof.

B14. The foregoing composition of B13, wherein the mutant Cas9 molecule comprises a D10A mutation.

B15. The foregoing composition of any one of B-B14, wherein the RNA-guided nuclease is a Cpf1 molecule.

B16. The foregoing composition of any one of B-B15, wherein the promoter is activated by a transactivator of a genome of the family, genus, or species of the targeted virus.

B17. The foregoing composition of B16, wherein the transactivator is selected from a group consisting of a HSV-1 ICP0 protein, a HSV-1 ICP4 protein, and a HSV-1 ICP27 protein.

C. In certain non-limiting embodiments, the presently disclosed subject matter provides a vector comprising a polynucleotide encoding: (a) an RNA-guided nuclease, and (b) a gRNA molecule comprising a targeting domain that is complementary with a target sequence of a target gene of a virus; wherein the expression of (a) and/or (b) is regulated by a promoter that is derived from a genome of the virus.

C1. The foregoing vector of C, wherein the promoter is derived from a gene of the family, genus, or species of the targeted virus.

C2. The foregoing vector of C or C1, wherein the promoter is operably linked to a polynucleotide encoding (a), and/or a polynucleotide encoding (b).

C3. The foregoing vector of any one of C-C2, wherein the promoter is derived from an immediate early gene, an early gene, or a late gene of the family, genus, or species of the targeted virus.

C4. The foregoing vector of any one of C-C3, wherein the expression of (a) and/or (b) is weak during a viral latency.

C5. The foregoing vector of any one of C-C4, wherein the expression of (a) and/or (b) is strong during a viral reactivation.

C6. The foregoing vector of C5, wherein the expression of (a) and/or (b) during the viral latency is at a level at least about 10%, at least about 20%, at least about 30%, at least about 40% or at least about 50% lower than the expression of (a) and/or (b) during the viral reactivation.

C7. The foregoing vector of any one of C-C6, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Eppstein-Barr virus, and a Kaposi's sarcoma-associated herpesvirus.

C8. The foregoing vector of any one of C-C7, wherein the targeted virus is selected from the group consisting of a Simplexvirus, a Varicellovirus, a Cytomegalovirus, a Roseolovirus, a Lymphocryptovirus, and a Rhadinovirus.

C9. The foregoing vector of C-C8, wherein the targeted virus is a Herpes Simplex Virus (HSV).

C10. The foregoing vector of any one of C-C9, wherein the targeted virus is a Herpes Simplex Virus-1 (HSV-1).

C11. The foregoing vector of any one of C-C10, wherein the RNA-guided nuclease is a Cas9 molecule.

C12. The foregoing vector of C11, wherein the Cas9 molecule comprises an S. pyogenes Cas9 molecule or an S. aureus Cas9 molecule.

C13. The foregoing vector of C11 or C12, wherein the Cas9 molecule comprises a wild-type Cas9 molecule, a mutant Cas9 molecule, or a combination thereof.

C14. The foregoing vector of C13, wherein the mutant Cas9 molecule comprises a D10A mutation.

C15. The foregoing vector of any one of C-C14, wherein the RNA-guided nuclease is a Cpf1 molecule.

C16. The foregoing vector of any one of C-C15, wherein the promoter is activated by a transactivator of the genome of the family, genus, or species of the targeted virus.

C17. The foregoing vector of C16, wherein the transactivator is selected from a group consisting of a HSV-1 ICP0 protein, a HSV-1 ICP4 protein, and a HSV-1 ICP27 protein.

D. In certain non-limiting embodiments, the presently disclosed subject matter provides a method of altering a target gene of a targeted virus in a cell, comprising administrating to the cell one of: (i) a genome editing system comprising a gRNA molecule comprising a targeting domain that is complementary with a target sequence of a target gene of the targeted virus, and an RNA-guided nuclease; (ii) a genome editing system comprising a polynucleotide encoding the gRNA molecule comprising the targeting domain that is complementary with the target sequence of the target gene of the targeted virus, and a polynucleotide encoding the RNA-guided nuclease; (iii) a composition comprising the gRNA molecule comprising the targeting domain that is complementary with the target sequence of the target gene of the targeted virus, and the RNA-guided nuclease; and (iv) a vector comprising a polynucleotide encoding the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

D1. The foregoing method of D, wherein the cell is an erythroid cell, or a trigeminal cell.

D2. The foregoing method of D or D1, wherein one of (i)-(iv) is administered in vivo.

D3. The foregoing method of any one of D-D2, wherein the promoter is derived from a gene of the family, genus, or species of the targeted virus.

D4. The foregoing method of any one of D-D3, wherein the promoter is operably linked to a polynucleotide encoding the gRNA molecule, and/or a polynucleotide encoding the RNA-guided nuclease.

D5. The foregoing method of any one of D-D4, wherein the promoter is derived from an immediate early gene, an early gene, or a late gene of the family, genus, or species of the targeted virus.

D6. The foregoing method of any one of D-D5, wherein the expression of the gRNA molecule and/or the RNA-guided nuclease is weak during a viral latency.

D7. The foregoing method of any one of D-D6, wherein the expression of the gRNA molecule and/or the RNA-guided nuclease is strong during a viral reactivation.

D8. The foregoing method of D7, wherein the expression of the gRNA molecule and/or the RNA-guided nuclease during the viral latency is at a level at least about 10%, at least about 20%, at least about 30%, at least about 40% or at least about 50% lower than the expression of the gRNA molecule and/or the RNA-guided nuclease during the viral reactivation.

D9. The foregoing method of any one of D-D8, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Epstein-Barr virus, and a Kaposi's sarcoma-associated herpesvirus.

D10. The foregoing method of D-D9, wherein the targeted virus is selected from the group consisting of a Simplexvirus, a Varicellovirus, a Cytomegalovirus, a Roseolovirus, a Lymphocryptovirus, and a Rhadinovirus.

D11. The foregoing method of any one of D-D10, wherein the targeted virus is a Herpes Simplex Virus (HSV).

D12. The foregoing method of any one of D-D11, wherein the targeted virus is a Herpes Simplex Virus-1 (HSV-1).

D13. The foregoing method of any one of D-D12, wherein the RNA-guided nuclease is a Cas9 molecule.

D14. The foregoing method of D13, wherein the Cas9 molecule comprises an S. pyogenes Cas9 molecule or an S. aureus Cas9 molecule.

D15. The foregoing method of D13 or D14, wherein the Cas9 molecule comprises a wild-type Cas9 molecule, a mutant Cas9 molecule, or a combination thereof.

D16. The foregoing method of D15, wherein the mutant Cas9 molecule comprises a D10A mutation.

D17. The foregoing method of any one of D-D16, wherein the RNA-guided nuclease is a Cpf1 molecule.

D18. The foregoing method of any one of D-D17, wherein the promoter is activated by a transactivator of a genome of the family, genus, or species of the targeted virus.

D19. The foregoing method of D18, wherein the transactivator is selected from a group consisting of a HSV-1 ICP0 protein, a HSV-1 ICP4 protein, and a HSV-1 ICP27 protein.

E. In certain non-limiting embodiments, the presently disclosed subject matter provides a method for treating and/or preventing a virus-related disease in a subject, comprising administrating to the subject one of: (i) a genome editing system comprising a gRNA molecule comprising a targeting domain that is complementary with a target sequence of a target gene of the targeted virus, and an RNA-guided nuclease; (ii) a genome editing system comprising a polynucleotide encoding the gRNA molecule comprising the targeting domain that is complementary with the target sequence of the target gene of the targeted virus, and a polynucleotide encoding the RNA-guided nuclease; (iii) a composition comprising the gRNA molecule comprising the targeting domain that is complementary with the target sequence of the target gene of the targeted virus, and the RNA-guided nuclease; and (iv) a vector comprising a polynucleotide encoding the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

E1. The foregoing method of E, wherein the promoter is derived from a gene of the family, genus, or species of the targeted virus.

E2. The foregoing method of E or E1, wherein the promoter is operably linked to a polynucleotide encoding the gRNA molecule, and/or a polynucleotide encoding the RNA-guided nuclease.

E3. The foregoing method of any one of E-E2, wherein the promoter is derived from an immediate early gene, an early gene, or a late gene of the family, genus, or species of the targeted virus.

E4. The foregoing method of any one of E-E3, wherein the expression of the gRNA molecule and/or the RNA-guided nuclease is weak during a viral latency.

E5. The foregoing method of any one of E-E4, wherein the expression of the gRNA molecule and/or the RNA-guided nuclease is strong during a viral reactivation.

E6. The foregoing method of E5, wherein the expression of the gRNA molecule and/or the RNA-guided nuclease during the viral latency is at a level at least about 10%, at least about 20%, at least about 30%, at least about 40% or at least about 50% lower than the expression of the gRNA molecule and/or the RNA-guided nuclease during the viral reactivation.

E7. The foregoing method of any one of E-E6, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Eppstein-Barr virus, and a Kaposi's sarcoma-associated herpesvirus.

E8. The foregoing method of any one of E-E7, wherein the targeted virus is selected from the group consisting of a Simplexvirus, a Varicellovirus, a Cytomegalovirus, a Roseolovirus, a Lymphocryptovirus, and a Rhadinovirus.

E9. The foregoing method of any one of E-E8, wherein the targeted virus is a Herpes Simplex Virus (HSV).

E10. The foregoing method of any one of E-E9, wherein the targeted virus is a Herpes Simplex Virus-1 (HSV-1).

E11. The foregoing method of any one of E-E10, wherein the RNA-guided nuclease is a Cas9 molecule.

E12. The foregoing method of E11, wherein the Cas9 molecule comprises an S. pyogenes Cas9 molecule or an S. aureus Cas9 molecule.

E13. The foregoing method of E11 or E12, wherein the Cas9 molecule comprises a wild-type Cas9 molecule, a mutant Cas9 molecule, or a combination thereof.

E14. The foregoing method of E13, wherein the mutant Cas9 molecule comprises a D10A mutation.

E15. The foregoing method of any one of E-E14, wherein the RNA-guided nuclease is a Cpf1 molecule.

E16. The foregoing method of any one of E-E15, wherein the promoter is activated by a transactivator of a genome of the family, genus, or species of the targeted virus.

E17. The foregoing method of E16, wherein the transactivator is selected from a group consisting of a HSV-1 ICP0 protein, a HSV-1 ICP4 protein, and a HSV-1 ICP27 protein.

E18. The foregoing method of any one of E-E17, wherein the administration is initiated at an early stage, a late stage, an advanced stage, a severe stage, or an acute stage of the viral-related disease.

E19. The foregoing method of any one of E-E18, wherein the administration is initiated prior to the subject is exposed to the targeted virus.

E20. The foregoing method of any one of E-E19, wherein the administration is initiated prior to the virus-related disease onset.

E21. The foregoing method of any one of E-E20, wherein the viral-related disease is a HSV-1 infection.

E22. The foregoing method of any one of E-E21, wherein the subject is a human subject.

F. In certain non-limiting embodiments, the presently disclosed subject matter provides genome editing system, comprising: (a) an RNA-guided nuclease; and (b) a gRNA molecule comprising a targeting domain that is complementary with a target sequence of a target gene of a targeted virus; wherein when the genome editing system is introduced in a cell infected by the targeted virus, the expression of the gene editing system correlates with transcriptional activity of the targeted virus, and/or genome abundance of the targeted virus.

F1. The foregoing genome editing system of F, wherein the expression of (a) and/or (b) is regulated by a promoter that is derived from a genome of the family, genus, or species of the targeted virus.

F2. The foregoing genome editing system of F or F1, wherein the promoter is operably linked to a polynucleotide encoding (a), and/or a polynucleotide encoding (b).

F3. The foregoing genome editing system of any one of F-F2, wherein the promoter is derived from an immediate early gene, an early gene, or a late gene of the family, genus, or species of the targeted virus.

F4. The foregoing genome editing system of any one of F-F3, wherein the expression of (a) and/or (b) is weak during a viral latency.

F5. The foregoing genome editing system of any one of F-F4, wherein the expression of (a) and/or (b) is strong during a viral reactivation.

F6. The foregoing genome editing system of F5, wherein the expression of (a) and/or (b) during the viral latency is at a level at least about 10%, at least about 20%, at least about 30%, at least about 40% or at least about 50% lower than the expression of (a) and/or (b) during the viral reactivation.

F7. The foregoing genome editing system of any one of F-F6, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Epstein-Barr virus, and a Kaposi's sarcoma-associated herpesvirus.

F8. The foregoing genome editing system of any one of F-F7, wherein the targeted virus is selected from the group consisting of a Simplexvirus, a Varicellovirus, a Cytomegalovirus, a Roseolovirus, a Lymphocryptovirus, and a Rhadinovirus.

F9. The foregoing genome editing system of any one of F-F8, wherein the targeted virus is a Herpes Simplex Virus (HSV).

F10. The foregoing genome editing system of any one of F-F9, wherein the targeted virus is a Herpes Simplex Virus-1 (HSV-1).

F11. The foregoing genome editing system of any one of F-F10, wherein the RNA-guided nuclease is a Cas9 molecule.

F12. The foregoing genome editing system of F11, wherein the Cas9 molecule comprises an S. pyogenes Cas9 molecule or an S. aureus Cas9 molecule.

F13. The foregoing genome editing system of F11 or F12, wherein the Cas9 molecule comprises a wild-type Cas9 molecule, a mutant Cas9 molecule, or a combination thereof.

F14. The foregoing genome editing system of F13, wherein the mutant Cas9 molecule comprises a D10A mutation.

F15. The foregoing genome editing system of any one of F-F14, wherein the RNA-guided nuclease is a Cpf1 molecule.

F16. The foregoing genome editing system of any one of F-F15, wherein the promoter is activated by a transactivator of a genome of the family, genus, or species of the targeted virus.

F17. The foregoing genome editing system of F16, wherein the transactivator is selected from a group consisting of a HSV-1 ICP0 protein, a HSV-1 ICP4 protein, and a HSV-1 ICP27 protein.

G. In certain non-limiting embodiments, the presently disclosed subject matter provides a genome editing system for use in altering a target gene of a targeted virus in a cell, wherein the genome editing system comprising: (i) a gRNA molecule comprising a targeting domain that is complementary with a target sequence of a target gene of the targeted virus, and an RNA-guided nuclease; or (ii) a polynucleotide encoding the gRNA molecule comprising the targeting domain that is complementary with the target sequence of the target gene of the targeted virus, and a polynucleotide encoding the RNA-guided nuclease, wherein the expression of the gRNA molecule and/or the RNA-guided nuclease is regulated by a promoter that is derived from a genome of the family, genus, or species of the targeted virus.

G1. In certain non-limiting embodiments, the presently disclosed subject matter provides a composition for use in altering a target gene of a targeted virus in a cell, wherein the composition comprising the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

G2. In certain non-limiting embodiments, the presently disclosed subject matter provides a vector for use in altering a target gene of a targeted virus in a cell, wherein the vector comprising a polynucleotide encoding the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

G3. The foregoing genome editing system of G, the foregoing composition of G1, or the foregoing vector of G2, wherein the cell is an erythroid cell, or a trigeminal cell.

G4. The foregoing genome editing system of G and G3, the foregoing composition of G1 and G3, or the foregoing vector of G2 and G3, wherein genome editing system, the composition, or the vector is administered in vivo.

G5. The foregoing genome editing system of any one of G, G3 and G4, the foregoing composition of any one of G1, G3 and G4, or the foregoing vector of any one of G2-G4, wherein the promoter is derived from a gene of the family, genus, or species of the targeted virus.

G6. The foregoing genome editing system of any one of G and G3-G5, the foregoing composition of any one of G1 and G3-G5, or the foregoing vector of any one of G2-G5, wherein the promoter is operably linked to a polynucleotide encoding the gRNA molecule, and/or a polynucleotide encoding the RNA-guided nuclease.

G7. The foregoing genome editing system of any one of G and G3-G6, the foregoing composition of any one of G1 and G3-G6, or the foregoing vector of any one of G2-G6, wherein the promoter is derived from an immediate early gene, an early gene, or a late gene of the family, genus, or species of the targeted virus.

G8. The foregoing genome editing system of any one of G and G3-G7, the foregoing composition of any one of G1 and G3-G7, or the foregoing vector of any one of G2-G7, wherein the expression of the gRNA molecule and/or the RNA-guided nuclease is weak during a viral latency.

G9. The foregoing genome editing system of any one of G and G3-G8, the foregoing composition of any one of G1 and G3-G8, or the foregoing vector of any one of G2-G8, wherein the expression of the gRNA molecule and/or the RNA-guided nuclease is strong during a viral reactivation.

G10. The foregoing genome editing system of any one of G and G3-G9, the foregoing composition of any one of G1 and G3-G9, or the foregoing vector of any one of G2-G9, wherein the expression of the gRNA molecule and/or the RNA-guided nuclease during the viral latency is at a level at least about 10%, at least about 20%, at least about 30%, at least about 40% or at least about 50% lower than the expression of the gRNA molecule and/or the RNA-guided nuclease during the viral reactivation.

G11. The foregoing genome editing system of any one of G and G3-G10, the foregoing composition of any one of G1 and G3-G10, or the foregoing vector of any one of G2-G10, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Epstein-Barr virus, and a Kaposi's sarcoma-associated herpesvirus.

G12. The foregoing genome editing system of any one of G and G3-G11, the foregoing composition of any one of G1 and G3-G11, or the foregoing vector of any one of G2-G11, wherein the targeted virus is selected from the group consisting of a Simplexvirus, a Varicellovirus, a Cytomegalovirus, a Roseolovirus, a Lymphocryptovirus, and a Rhadinovirus.

G13. The foregoing genome editing system of any one of G and G3-G12, the foregoing composition of any one of G1 and G3-G12, or the foregoing vector of any one of G2-G12, wherein the targeted virus is a Herpes Simplex Virus (HSV).

G14. The foregoing genome editing system of any one of G and G3-G13, the foregoing composition of any one of G1 and G3-G13, or the foregoing vector of any one of G2-G13, wherein the targeted virus is a Herpes Simplex Virus-1 (HSV-1).

G15. The foregoing genome editing system of any one of G and G3-G14, the foregoing composition of any one of G1 and G3-G14, or the foregoing vector of any one of G2-G14, wherein the RNA-guided nuclease is a Cas9 molecule.

G16. The genome editing system, the composition, or the vector of G15, wherein the Cas9 molecule comprises an S. pyogenes Cas9 molecule or an S. aureus Cas9 molecule.

G17. The genome editing system, the composition, or the vector of G15 or G16, wherein the Cas9 molecule comprises a wild-type Cas9 molecule, a mutant Cas9 molecule, or a combination thereof.

G18. The genome editing system, the composition, or the vector of G17, wherein the mutant Cas9 molecule comprises a D10A mutation.

G19. The foregoing genome editing system of any one of G and G3-G18, the foregoing composition of any one of G1 and G3-G18, or the foregoing vector of any one of G2-G18, wherein the RNA-guided nuclease is a Cpf1 molecule.

G20. The foregoing genome editing system of any one of G and G3-G19, the foregoing composition of any one of G1 and G3-G19, or the foregoing vector of any one of G2-G19, wherein the promoter is activated by a transactivator of a genome of the family, genus, or species of the targeted virus.

G21. The genome editing system, the composition, or the vector of G20, wherein the transactivator is selected from a group consisting of a HSV-1 ICP0 protein, a HSV-1 ICP4 protein, and a HSV-1 ICP27 protein.

H. In certain non-limiting embodiments, the presently disclosed subject matter provides a genome editing system for use in treating and/or preventing a virus-related disease in a subject, wherein the genome editing system comprising: (i) a gRNA molecule comprising a targeting domain that is complementary with a target sequence of a target gene of the targeted virus, and an RNA-guided nuclease; or (ii) a polynucleotide encoding the gRNA molecule comprising the targeting domain that is complementary with the target sequence of the target gene of the targeted virus, and a polynucleotide encoding the RNA-guided nuclease, wherein the expression of the gRNA molecule and/or the RNA-guided nuclease is regulated by a promoter that is derived from a genome of the family, genus, or species of the targeted virus.

H1. In certain non-limiting embodiments, the presently disclosed subject matter provides a composition for use in treating and/or preventing a virus-related disease in a subject, wherein the composition comprising the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

H2. In certain non-limiting embodiments, the presently disclosed subject matter provides a vector for use in treating and/or preventing a virus-related disease in a subject, wherein the vector comprising a polynucleotide encoding the gRNA molecule comprising the targeting domain that is complementary with the target sequence of the target gene of the targeted virus, and the RNA-guided nuclease, wherein the expression of the gRNA molecule and/or the RNA-guided nuclease is regulated by a promoter that is derived from a genome of the family, genus, or species of the targeted virus.

H3. The genome editing system of H, the composition of H1, or the vector of H2, wherein the promoter is derived from a gene of the family, genus, or species of the targeted virus.

H4. The genome editing system of H or H3, the composition of H1 or H3, or the vector of H2 or H3, wherein the promoter is operably linked to a polynucleotide encoding the gRNA molecule, and/or a polynucleotide encoding the RNA-guided nuclease.

H5. The genome editing system of any one of H, H3 or H4, the composition of any one of H1, H3 or H4, or the vector of any one of H2-H4, wherein the promoter is derived from an immediate early gene, an early gene, or a late gene of the family, genus, or species of the targeted virus.

H6. The genome editing system of any one of H, H3-H5, the composition of any one of H1, H3-H5, or the vector of any one of H2-H5, wherein the expression of the gRNA molecule and/or the RNA-guided nuclease is weak during a viral latency.

H7. The genome editing system of any one of H, H3-H6, the composition of any one of H1, H3-H6, or the vector of any one of H2-H6, wherein the expression of the gRNA molecule and/or the RNA-guided nuclease is strong during a viral reactivation.

H8. The genome editing system of any one of H, H3-H7, the composition of any one of H1, H3-H7, or the vector of any one of H2-H7, wherein the expression of the gRNA molecule and/or the RNA-guided nuclease during the viral latency is at a level at least about 10%, at least about 20%, at least about 30%, at least about 40% or at least about 50% lower than the expression of the gRNA molecule and/or the RNA-guided nuclease during the viral reactivation.

H9. The genome editing system of any one of H, H3-H8, the composition of any one of H1, H3-H8, or the vector of any one of H2-H8, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Epstein-Barr virus, and a Kaposi's sarcoma-associated herpesvirus.

H10. The genome editing system of any one of H, H3-H9, the composition of any one of H1, H3-H9, or the vector of any one of H2-H9, wherein the targeted virus is selected from the group consisting of a Simplexvirus, a Varicellovirus, a Cytomegalovirus, a Roseolovirus, a Lymphocryptovirus, and a Rhadinovirus.

H11. The genome editing system of any one of H, H3-H10, the composition of any one of H1, H3-H10, or the vector of any one of H2-H10, wherein the targeted virus is a Herpes Simplex Virus (HSV).

H12. The genome editing system of any one of H, H3-H11, the composition of any one of H1, H3-H11, or the vector of any one of H2-H11, wherein the targeted virus is a Herpes Simplex Virus-1 (HSV-1).

H13. The genome editing system of any one of H, H3-H12, the composition of any one of H1, H3-H12, or the vector of any one of H2-H12, wherein the RNA-guided nuclease is a Cas9 molecule.

H14. The genome editing system, the composition, or the vector of H13, wherein the Cas9 molecule comprises an S. pyogenes Cas9 molecule or an S. aureus Cas9 molecule.

H15. The genome editing system, the composition, or the vector of H13 or H14, wherein the Cas9 molecule comprises a wild-type Cas9 molecule, a mutant Cas9 molecule, or a combination thereof.

H16. The genome editing system, the composition, or the vector of H15, wherein the mutant Cas9 molecule comprises a D10A mutation.

H17. The genome editing system of any one of H, H3-H16, the composition of any one of H1, H3-H16, or the vector of any one of H2-H16, wherein the RNA-guided nuclease is a Cpf1 molecule.

H18. The genome editing system of any one of H, H3-H17, the composition of any one of H1, H3-H17, or the vector of any one of H2-H17, wherein the promoter is activated by a transactivator of a genome of the family, genus, or species of the targeted virus.

H19. The genome editing system, the composition, or the vector of H18, wherein the transactivator is selected from a group consisting of a HSV-1 ICP0 protein, a HSV-1 ICP4 protein, and a HSV-1 ICP27 protein.

H20. The genome editing system of any one of H, H3-H19, the composition of any one of H1, H3-H19, or the vector of any one of H2-H19, wherein the administration is initiated at an early stage, a late stage, an advanced stage, a severe stage, or an acute stage of the viral-related disease.

H21. The genome editing system of any one of H, H3-H20, the composition of any one of H1, H3-H20, or the vector of any one of H2-H20, wherein the administration is initiated prior to the subject is exposed to the targeted virus.

H22. The genome editing system of any one of H, H3-H21, the composition of any one of H1, H3-H21, or the vector of any one of H2-H21, wherein the administration is initiated prior to the virus-related disease onset.

H23. The genome editing system of any one of H, H3-H22, the composition of any one of H1, H3-H22, or the vector of any one of H2-H22, wherein the viral-related disease is a HSV-1 infection.

H24. The genome editing system of any one of H, H3-H23, the composition of any one of H1, H3-H23, or the vector of any one of H2-H23, wherein the subject is a human subject.

### EXAMPLES

The following Examples are merely illustrative and are not intended to limit the scope or content of the invention in any way.

### Example 1: Selection of HSV Promoters to be Used with the Presently Disclosed Gene Editing Systems

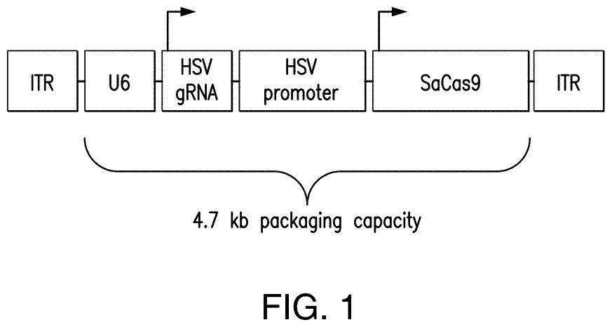

AAV vectors were designed to encode CRISPR/Cas system for targeting HSV (FIG. 1). The vector included: Inverted terminal repeats (ITR), U6 promoter driving guide RNA (gRNA) expression that targeted HSV genomic sequences, and an HSV-dependent promoter derived from the HSV genome driving the expression of SaCas9. If the promoter was small enough, the vector could accommodate two U6-HSV gRNA expression cassettes maximum, given the 4.7 kb packaging limit.

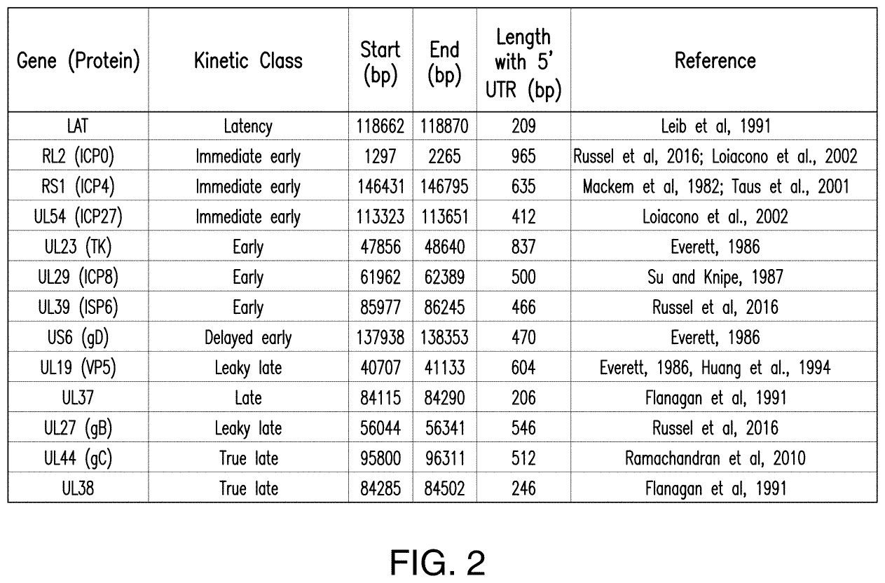

A literature search was performed to identify HSV promoters which have been either defined by sequence or used for heterologous gene expression (FIG. 2). The HSV promoter sequences SEQ ID NOs: 1-14 were pulled from the literature search and identified within the published HSV-1 stain 17+ genomic DNA sequence (NCBI accession JN555585.1). Boundaries were identified within the literature identified in FIG. 2 and promoter sequences all the way up to the start codon of the respective open reading frame (ORF) were added as to include any 5′ untranslated region (UTR) elements. Promoters were selected from each of the unique viral expression kinetic classes, including immediate-early (IE), early (E), and late (L) promoters.

### Example 2: Characterization of the Selected HSV-1 Promoters

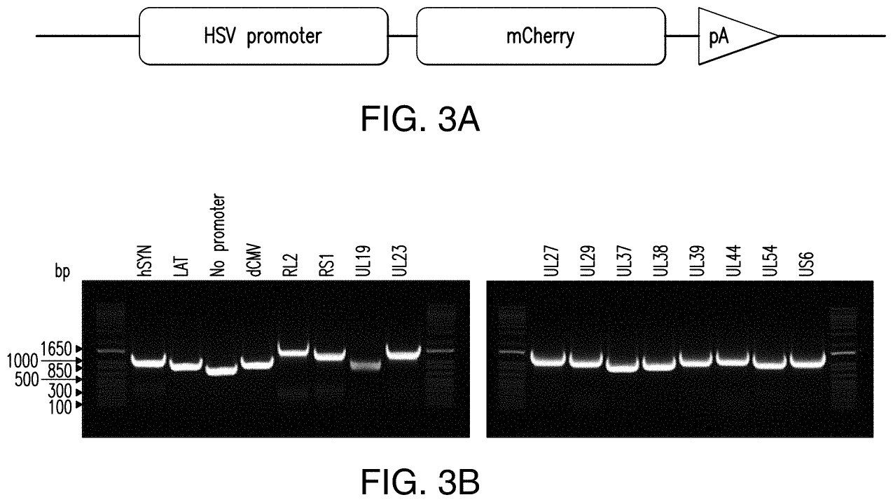

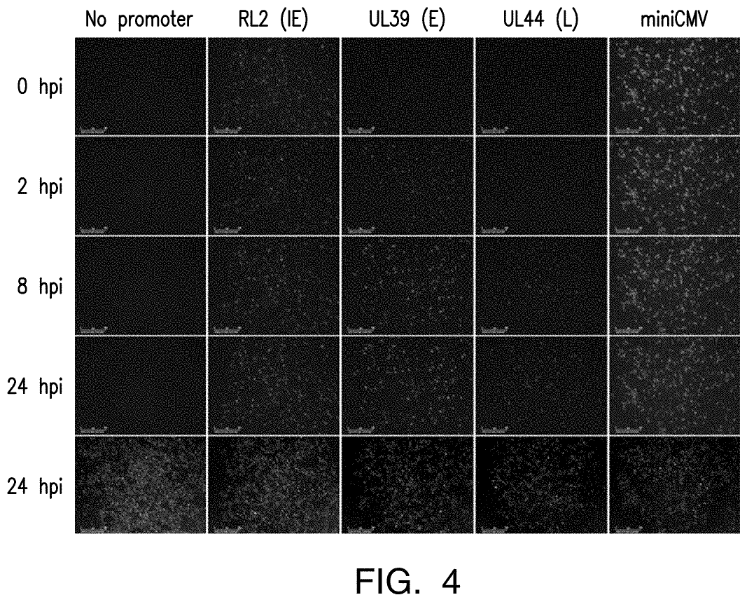

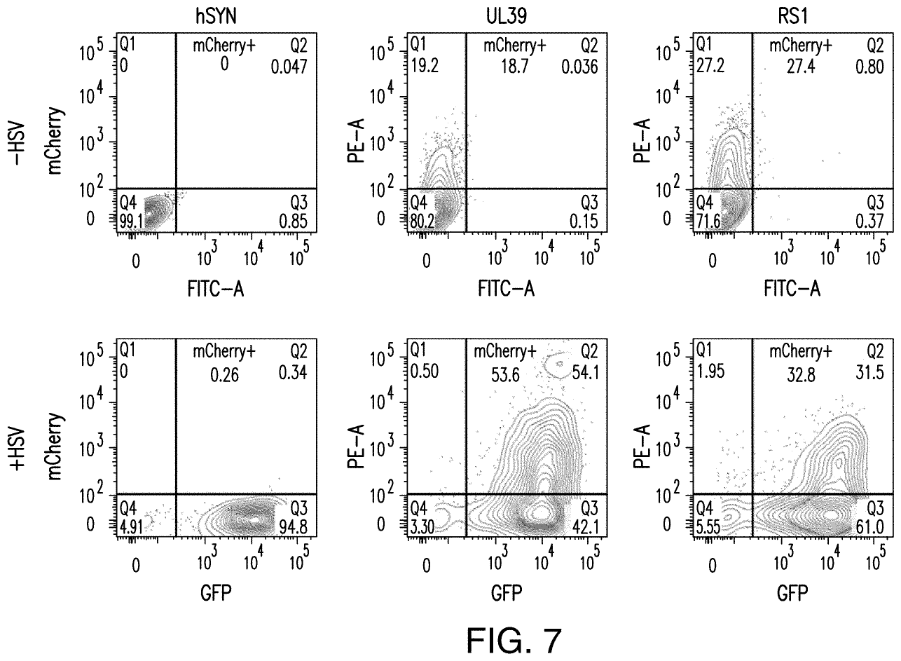

Promoters selected from Example 1 were cloned from HSV-1 17+ genomic DNA isolated from infected cells and fused to an mCherry reporter gene with mini poly(A) tail using PCR then overlap extension PCR (FIGS. 3A-3B). Each HSV promoter-mCherry amplicon was then cloned into pUC19 and confirmed by Sanger sequencing. Vero cell cultures were nucleofected with each HSV promoter-mCherry expression cassette and then mock- or HSV-1-infected (HSV-1-GFP). Flow cytometry analysis confirmed that HSV promoters were inducible by HSV-infection (FIG. 7). Cells were imaged by fluorescence microscopy over the course of the 24 hours post-infection. The expression characteristics of promoters belonging to each of the three gene kinetic class are captured by microscopy (FIG. 4). IE gene promoters such as RL2 were constitutively expressed, independent of HSV-1 infection. E and L gene promoters such as UL39 and UL44, respectively, were induced to express only during HSV-1 infection. E gene promoter UL39 expression was induced within the first 2 hours of infection, achieving maximal sustained expression strength by 8 hours post-infection. In contrast, L gene promoter UL44 expression was not achieved until 8 hours post-infection.

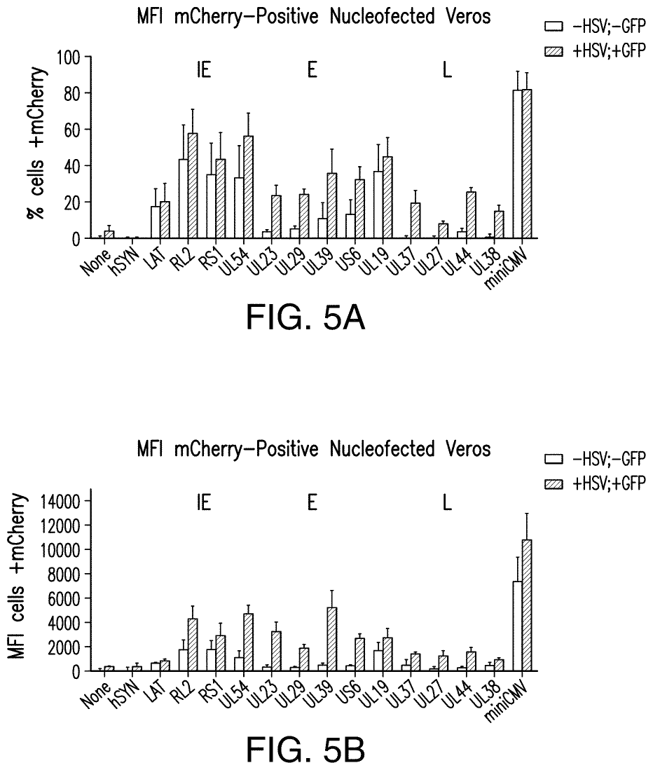

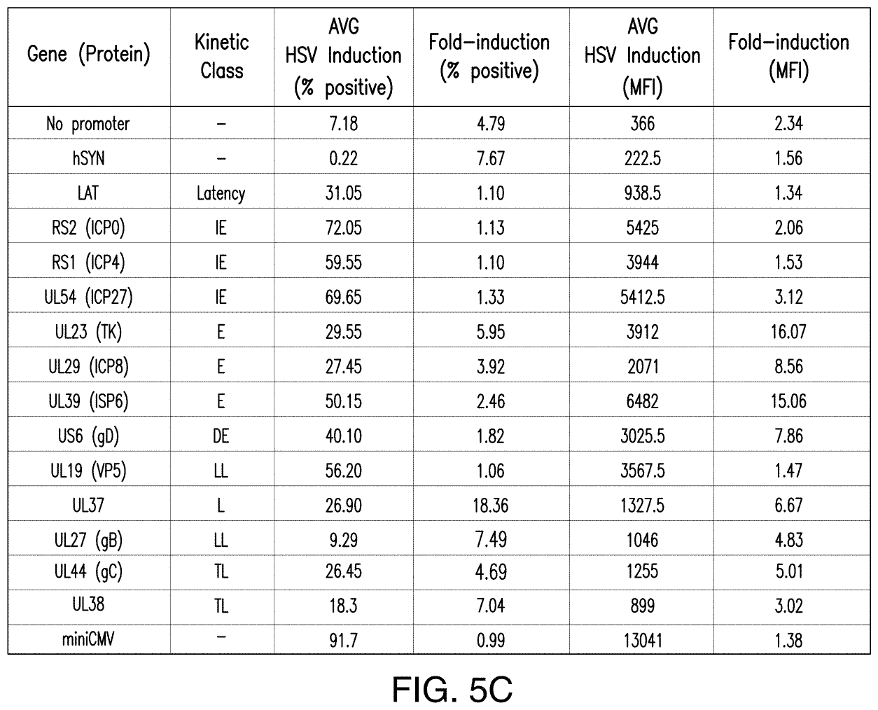

To quantify promoter strength and HSV-dependency, Vero cells were again nucleofected with HSV promoter-mCherry expression cassettes and mock- or HSV-1-infected (HSV-1-GFP) for 8 hours. Cells were fixed and analyzed by flow cytometry to quantify mCherry positivity and mCherry mean fluorescence intensity (MFI) in GFP-negative (uninfected) or GFP-positive (HSV-1-infected) cells (FIGS. 5A-5C). Of the tested HSV promoters, IE promoters (RL2, RS1, and UL54) had the highest levels of expression across cell populations in terms of both percent mCherry-positivity (comparable to positive miniCMV control promoter) and MFI. However, IE promoter expression was not induced by HSV-1 infection, indicating these promoters are constitutively active and not dependent upon HSV-1 infection. E gene promoters (UL23, UL29, UL39, US6) had mid-range levels of maximal MFI compared to the positive control miniCMV promoter, but demonstrated the highest levels HSV-1-induced MFI of all tested promoters. E promoters exhibited mCherry expression in roughly 50% of cell population. L gene promoters (UL19, UL37, UL27, UL44, UL38), with the exception of UL19, showed the lowest levels of mCherry positivity and maximal MFI, but high HSV-1-inducibility overall. Per flow cytometry analysis, several of these promoters demonstrated the ideal characteristics for delivering SaCas9 to HSV-1-infected cells in an HSV-1-dependent manner, including: 1) Significant fraction of cell population basally expressing mCherry; and 2) Strong inducibility of mCherry MFI upon HSV-1 infection

These two characteristics ensure that cells would have a low, but appreciable, amount of SaCas9 prior to infection and see a significant induction of SaCas9 only upon HSV-1 infection. Exemplary promoters demonstrating these characteristics include all E promoters UL23, UL29, UL39, and US6 and IE promoter UL54.

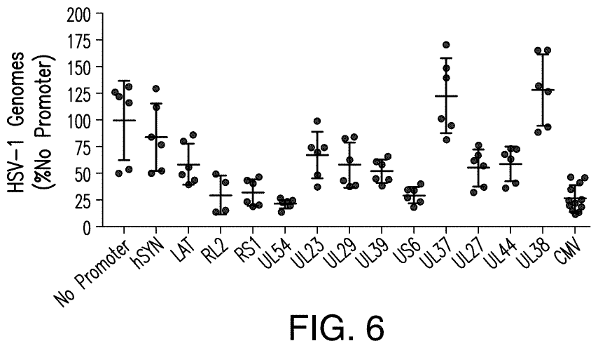

To test whether these promoters could actually provide CRISPR/Cas-based protection within a cell culture model, each HSV promoter was fused to SaCas9 cDNA and cloned into pUC19 vector along with an expression cassette for a UL48-targeting gRNA driven by a U6 promoter. Vero cells were nucleofected and challenged with HSV. After 24 hours of infection, HSV-1 genomes present within the cell culture supernatant were quantified by qPCR (FIG. 6). Relative to the “No promoter” control, IE gene promoters RL2, RS1, and UL54, and E gene promoter US6 provided a ˜75% knockdown effect of HSV-1, comparable to the positive CMV control promoter. This indicates that these promoters were able to induce enough expression of SaCas9 to significantly impact the replication and spread of HSV-1 in culture. This is consistent with RL2 and RS1 achieving high levels of transgene expression, independent of HSV-1 infection, within the cell culture. Additionally, UL54 and US6 were highly HSV-inducible based on our mCherry reporter studies. Thus, UL54 and US6 are prime candidate promoters for HSV-dependent delivery of SaCas9 to HSV-infected cells.

### REFERENCES

Leib, D. A., et al., The promoter of the latency-associated transcripts of herpes simplex virus type 1 contains a functional cAMP-response element: role of the latency-associated transcripts and cAMP in reactivation of viral latency. Proc Natl Acad Sci USA, 1991. 88(1): p. 48-52.
Loiacono, C. M., R. Myers, and W. J. Mitchell, Neurons differentially activate the herpes simplex virus type 1 immediate-early gene ICP0 and ICP27 promoters in transgenic mice. J Virol, 2002. 76(5): p. 2449-59.
Russell, T. A. and D. C. Tscharke, Lytic Promoters Express Protein during Herpes Simplex Virus Latency. PLoS Pathog, 2016. 12(6): p. e1005729.
Mackem, S. and B. Roizman, Structural features of the herpes simplex virus alpha gene 4, 0, and 27 promoter-regulatory sequences which confer alpha regulation on chimeric thymidine kinase genes. J Virol, 1982. 44(3): p. 939-49.
Taus, N. S. and W. J. Mitchell, The transgenic ICP4 promoter is activated in Schwann cells in trigeminal ganglia of mice latently infected with herpes simplex virus type 1. J Virol, 2001. 75(21): p. 10401-8.
Loiacono, C. M., R. Myers, and W. J. Mitchell, Neurons differentially activate the herpes simplex virus type 1 immediate-early gene ICP0 and ICP27 promoters in transgenic mice. J Virol, 2002. 76(5): p. 2449-59.
Everett, R. D., The products of herpes simplex virus type 1 (HSV-1) immediate early genes 1, 2 and 3 can activate HSV-1 gene expression in trans. J Gen Virol, 1986. 67 (Pt 11): p. 2507-13.
Su, L. and D. M. Knipe, Mapping of the transcriptional initiation site of the herpes simplex virus type 1 ICP8 gene in infected and transfected cells. J Virol, 1987. 61(2): p. 615-20.
Russell, T. A. and D. C. Tscharke, Lytic Promoters Express Protein during Herpes Simplex Virus Latency. PLoS Pathog, 2016. 12(6): p. e1005729.
Everett, R. D., The products of herpes simplex virus type 1 (HSV-1) immediate early genes 1, 2 and 3 can activate HSV-1 gene expression in trans. J Gen Virol, 1986. 67 (Pt 11): p. 2507-13.
Huang, C. J. and E. K. Wagner, The herpes simplex virus type 1 major capsid protein (VP5-UL19) promoter contains two cis-acting elements influencing late expression. J Virol, 1994. 68(9): p. 5738-47.
Flanagan, W. M., et al., Analysis of the herpes simplex virus type 1 promoter controlling the expression of UL38, a true late gene involved in capsid assembly. J Virol, 1991. 65(2): p. 769-86.
Ramachandran, S., et al., Delaying the expression of herpes simplex virus type 1 glycoprotein B (gB) to a true late gene alters neurovirulence and inhibits the gB-CD8+ T-cell response in the trigeminal ganglion. J Virol, 2010. 84(17): p. 8811-20.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

1. A genome editing system comprising:
(a) an RNA-guided nuclease, and
(b) a gRNA molecule comprising a targeting domain that is complementary with a target sequence of a target gene of a targeted virus;
wherein the expression of (a) and/or (b) is regulated by a promoter that is derived from a genome of the family, genus, or species of the targeted virus.

2. The genome editing system of claim 1, wherein the promoter (i) is operably linked to a polynucleotide encoding, and/or a polynucleotide encoding (b); and/or (ii) is derived from an immediate early gene, an early gene, or a late gene of the family, genus, or species of the targeted virus.

3. The genome editing system of claim 1, wherein the expression of (a) and/or (b) (i) is weak during a viral latency; and/or (ii) is strong during a viral reactivation.

4. The genome editing system of claim 3, wherein the expression of (a) and/or (b) is strong during a viral reactivation, and the expression of (a) and/or (b) during the viral latency is at a level at least about 10%, at least about 20%, at least about 30%, at least about 40% or at least about 50% lower than the expression of (a) and/or (b) during the viral reactivation.

5. The genome editing system of claim 4, wherein the targeted virus is selected from the group consisting of a Herpesviridae, a Alphaherpesvirinae, a Betaherpesvirinae, and a Gammaherpesvirinae, an Iltovirus, a Mardivirus, a Simplexvirus, a Scutavirus, a Varicellovirus, Cytomegalovirus, a Morumegalovirus, a Proboscivirus, a Roseolovirus, a Lymphocryptovirus, a Macavirus, a Percavirus, a Rhadinovirus, an Epstein-Barr virus, and a Kaposi's sarcoma-associated herpesvirus.

6. The genome editing system of claim 1, wherein the targeted virus is a Herpes Simplex Virus (HSV), or a Herpes Simplex Virus-1 (HSV-1).

7. The genome editing system of claim 1, wherein the RNA-guided nuclease is a Cas9 molecule.

8. The genome editing system of claim 7, wherein the Cas9 molecule comprises an S. pyogenes Cas9 molecule, an S. aureus Cas9 molecule, a wild-type Cas9 molecule, a mutant Cas9 molecule, or a combination thereof.

9. The genome editing system of claim 8, wherein the mutant Cas9 molecule comprises a D10A mutation.

10. The genome editing system of claim 1, wherein the RNA-guided nuclease is a Cpf1 molecule.

11. The genome editing system of claim 1, wherein the promoter is activated by a transactivator of a genome of the family, genus, or species of the targeted virus.

12. The genome editing system of claim 11, wherein the transactivator is selected from a group consisting of a HSV-1 ICP0 protein, a HSV-1 ICP4 protein, and a HSV-1 ICP27 protein.

13. A composition comprising
(a) an RNA-guided nuclease, and
(b) a gRNA molecule comprising a targeting domain that is complementary with a target sequence of a target gene of a targeted virus;
wherein the expression of (a) and/or (b) is regulated by a promoter that is derived from a genome of the family, genus, or species of the targeted virus.

14. A vector comprising a polynucleotide encoding the genome editing system of claim 1.

15. A method of altering a target gene of a targeted virus in a cell, comprising administrating to the cell a genome editing system of claim 1.

16. The method of claim 15, wherein the cell is an erythroid cell, or a trigeminal cell.

17. A method for treating and/or preventing a virus-related disease in a subject, comprising administrating to the subject a genome editing system of claim 1.

18. The method of claim 17, wherein (i) the administration is initiated at an early stage, a late stage, an advanced stage, a severe stage, or an acute stage of the viral-related disease; (ii) the administration is initiated prior to the subject is exposed to the targeted virus; and/or (iii) the administration is initiated prior to the virus-related disease onset.

19. The method of claim 17, wherein the viral-related disease is a HSV-1 infection.

20. A genome editing system, comprising
(a) an RNA-guided nuclease; and
(b) a gRNA molecule comprising a targeting domain that is complementary with a target sequence of a target gene of a targeted virus;
wherein when the genome editing system is introduced in a cell infected by the targeted virus, the expression of the gene editing system correlates with transcriptional activity of the targeted virus, and/or genome abundance of the targeted virus.


SEQUENCE LISTING (14 sequences)

SEQ ID NO: 1 - DNA, 209 bp, Artificial Sequence
  Synthetic LAT promoter
ctgcagacaggggcaccgcgcccggaaatccattaggccgcagacgaggaaaataaaattacatcacctacccacgtggtgctgtggcctgtttttgctgcgtcatctcagcctttataaaagcgggggcgcggccgtgccgatcgcgggtggtgcgaaagactttccgggcgcgtccgggtgccgcggctctccgggcccccctgcag

SEQ ID NO: 2 - DNA, 965 bp, Artificial Sequence
  Synthetic RL2 promoter
cccgggagctccgcaccaagccgctctccggagagacgatggcaggagccgcgcatatatacgcttggagccagcccgccctcacagggcgggcccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgaccccc

SEQ ID NO: 3 - DNA, 635 bp, Artificial Sequence
  Synthetic RS1 promoter
cccgggccccgccccctgcccgttcctcgttagcatgcggaacggaagcggaaaccgccggatcgggcggtaatgagatgccatgcggggcggggcgcggacccacccgccctcgcgccccgcccatggcagatggcgcggatgggcggggccgggggttcgaccaacgggccgcggccacgggcccccggcgtgccggcgtcggggcggggtcgtgcataatggaattccgttcggggtgggcccgccgggggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttcccacccaagcatcgaccggtccgcgctagttccgcgtcgacggcgggggtcgtcggggtccgtgggtctcgccccctccccccatcgagagtccgtaggtgacctaccgtgctacgtccgccgtcgcagccgtatccccggaggatcgccccgcatcggcg

SEQ ID NO: 4 - DNA, 837 bp, Artificial Sequence
  Synthetic UL23 promoter
ggatccaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctcaatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaataccgagcgaccctgcagcgacccgcttaacagcgtcaacagcgtgccgcagatcttggtggcgtgaaactcccgcacctcttcggccagcgccttgtagaagcgcgt

SEQ ID NO: 5 - DNA, 546 bp, Artificial Sequence
  Synthetic UL27 promoter
tcaacgggcccctctttgatcactccacccacagcttcgcccagccccccaacaccgcgctgtattacagcgtcgagaacgtggggctcctgccgcacctgaaggaggagctcgcccggttcatcatgggggcggggggctcgggtgctgattgggccgtcagcgaatttcagaggttttactgttttgacggcatttccggaataacgcccactcagcgcgccgcctggcgatatattcgcgagctgattatcgccaccacactctttgcctcggtctaccggtgcggggagctcgagttgcgccgcccggactgcagccgcccgacctccgaaggtcgttaccgttacccgcccggcgtatatctcacgtacgactccgactgtccgctggtggccatcgtcgagagcgcccccgacggctgtatcggcccccggtcggtcgtggtctacgaccgagacgttttctcgatcctctactcggtcctccagcacctcgcccccaggctacctgacggggggcacgacgggcccccgtagtcccgcc

SEQ ID NO: 6 - DNA, 450 bp, Artificial Sequence
  Synthetic UL29 promoter
cgggcggcgagctgctgcgcggcgccccggccggcggcccggtttattcgcgtcggcccggccggccgggcttatggaccgccggcggccgacaggagagtgacgtagccggtgggcgtggaggggctggggcggaccggcacgcccccaggtaaagtgtacatataccaaccgcataccagacgcacccgacccggagcacctgaccgtaagcatctgtgcctctcgcagggaccccgcgttgccggccgccggggttcatcggcaccccgtggttacccggggggttgtcggtgaaggggagggattcattccccaaccccggtctccaaccctccccttgaccgtcgccgcccccccccggattttgacgctcgggagacataccttgtcgggcgtccgtcgtcgtgccgggattacctccgttcgcggaccgattgacaaaaggac

SEQ ID NO: 7 - DNA, 206 bp, Artificial Sequence
  Synthetic UL37 promoter
cccgggcctgggtccgcgaacgggatgccgggacttaagtggccgtataacaccccgcgaagacgcggggtactcgcaacgcctgcgggggtcctggagggccgcgggggatcgataattcgccgctccctacagcgcacgacagtcattcccgcccggtctcgtcgttggtctacgctgtcccccacccacgcgagccgggcgtc

SEQ ID NO: 8 - DNA, 246 bp, Artificial Sequence
  Synthetic UL38 promoter
cccgggggattgtccggatgtgcgggcagcccggacggcgtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgacccccccccccctccctcccccgggtcttgctaggtcgcgatctggggtcgca

SEQ ID NO: 9 - DNA, 466 bp, Artificial Sequence
  Synthetic UL39 promoter
ccgctgtcactcgttgttcgttgacccgggcgtccgccaaataaagccactgaaacccgaaacgcgagtgttgtaacgtcctttgggcgggaggaagccacaaaatgcaaatgggatacatggaaggaacacacccccgtgactcaggacatcggtgtgtccttttgggtttcactgaaactggcccgcgccccacccctgcgcgatgtggataaaaagccagcgcgggtggtttagggtaccacaggtgggtgctttggaaacttgccggtcgccgtgctcctgtgagcttgcgtccctccccggtttcctttgcgctcccgccttccggacctgctctcgcctatcttctttggctctcggtgcgattcgtcaggcagcggccttgtcgaatctcgaccccaccactcgccggacccgccgacgtcccctctcgagcccgccgaaacccgccgcgtctgttgaa

SEQ ID NO: 10 - DNA, 326 bp, Artificial Sequence
  Synthetic UL42 promoter
atttcgatggcccaactccacgcggattggtgcagcaccctgcatgcgccggtgcgggccaaccttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccatcaaaaccccaccctcgcagcggcatacgccccctccgcgtcccgcacccgagaccccggcccggctgccctcaccaccgaagcccacctcgtcactgtggggtgttcccagcccgcgttggg

SEQ ID NO: 11 - DNA, 512 bp, Artificial Sequence
  Synthetic UL44 promoter
gcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggtgctgctgtgccctccggggtcaacgggcgggcggtcgggcgattgatatatttttcaataaaaggcattagtcccgaagaccgccggtgtgtgatgatttcgccataacacccaaaccccggatggggcccgggtataaattccggaaggggacacgggctaccctcactatcgagggcgcttggtcgggaggccgcatcgaacgcacacccccatccggtggtccgtgtggaggtcgttttcagtgcccggtctcgctttgccgggaacgctagccgatccctcgcgagggggaggcgtcgggc

SEQ ID NO: 12 - DNA, 373 bp, Artificial Sequence
  Synthetic UL48 promoter
ggggttcattcggtgttggcgttgcgtgcctttgtttcccaatccgacggggaccgggactgggtggcggggggtgggttggacagccgccctcggttcgccttcacgtgacaggagccaatgtggggggaagtcacgaggtacggggcggcccgtgcgggttgcttaaatgcgtggtggcgaccacgggctgtcattcctcgggaacggacggggttcccgctgcccacttccccccataaggtccgtccggtcctctaacgcgtttgggggttttctcttcccgcgccgtcgggcgtcccacactctctgggcgggcggggacgatcgcatcaaaagcccgatatcgtctttcccgtatcaaccccaccca

SEQ ID NO: 13 - DNA, 401 bp, Artificial Sequence
  Synthetic UL54 promoter
accccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtc

SEQ ID NO: 14 - DNA, 485 bp, Artificial Sequence
  Synthetic US6 promoter
ctgcttgagctcctgcgtcgtacgtgccgcgggtgggggcgttaccatccctacatggacccagttgtcgtataatttcccccccccccccccttctccgcgtgggtgatgtcgggtccaaactcccgacaccaccagctggcatggtataaatcaccggtgcgccccccaaaccatgtccggcagggggatgggggggcgaatgcggagggcacccaacaacaccgggctaaccaggaaatccgtggccccggcccccaataaagatcgcggtagcccggccgtgtgacactatcgtccataccgaccacaccgacgaatcccccaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggt